(19) United States
(12) Patent Application Publication
(10) Pub. No.: US 2021/0003593 A1
(43) Pub. Date: Jan. 7, 2021

(54) CHITINASE PROTEINS IN NEUROLOGIC DISEASE

(71) Applicant: Dignity Health, San Francisco, CA (US)

(72) Inventors: Robert Bowser, San Francisco, CA (US); Lucas Vu, San Francisco, CA (US)

(21) Appl. No.: 16/975,877

(22) PCT Filed: Feb. 26, 2019
(86) PCT No.: PCT/US2019/019671

§ 371 Date: Aug. 26, 2020

Related U.S. Application Data
(60) Provisional application No. 62/634,984, filed on Feb. 26, 2018.
(60) Provisional application No. 62/639,273, filed on Mar. 6, 2018.

(51) Int. Cl. G01N 33/68

(52) U.S. Cl. CPC G01N 33/6896; C12Y 302/01014; G01N 2333/924; G01N 2800/28

(57) ABSTRACT

The present disclosure describes methods of determining a treatment protocol for and/or a prognosis for a subject suspected of or at risk of suffering from a neurologic disease or disorder, including such diseases and disorders that involve motor neuron function. In certain aspects, methods are provided for determining a concentration of a one or more chitinase proteins in a biological fluid sample containing or suspected of containing chitinase protein.

### CROSS-REFERENCE TO RELATED APPLICATIONS

This application claims the priority of U.S. provisional application No. 62/634,984, filed Feb. 26, 2018, and U.S. provisional application No. 62/639,273, filed Mar. 6, 2018, the disclosures of which are hereby incorporated by reference in their entireties.

### FIELD OF THE INVENTION

The present disclosure generally relates, in certain aspects, to methods of determining a treatment protocol for and/or a prognosis for a subject suspected of or at risk of suffering from a neurologic disease or disorder, including such diseases and disorders that involve motor neuron function. In certain aspects, methods are provided for determining a concentration of one or more chitinase proteins in a biological fluid sample.

### BACKGROUND OF THE INVENTION

Inflammation in the peripheral nervous system and central nervous system is a key element of many serious neurologic diseases for which diagnostic, prognostic and monitoring methods are generally lacking. Further, such diseases can be difficult to discriminate among the others that share clinical signs. For example, early detection and diagnosis of Amyotrophic lateral sclerosis (ALS) is difficult because its signs can be very similar to other neurologic diseases. Methods for diagnosing ALS currently include Electromyogram (EMG), nerve conduction study, magnetic resonance imaging (MRI), spinal tap (lumbar puncture) or muscle biopsy, but many of these tests only function to rule out other possibilities. Moreover, many of these tests are expensive, and/or invasive and painful.

Currently there exists no known marker for astrocyte or microglial activation in neurologic diseases. Current approaches to identify microglial activation in the brain generally focus on imaging techniques, which require access to expensive imaging facilities and generally are costly to obtain and interpret. Ideally, a test for astrocyte or microglial activation as an indicator of neurologic disease would not only be reliable, but could be performed noninvasively and inexpensively, would provide subjects with ready access to the test, readily distinguish astrocyte or microglial activation, stratify patients, predict and monitor disease progression, and where appropriate, focus therapy which targets glial activation. A need remains for such a test.

### SUMMARY OF THE INVENTION

In one aspect, the present disclosure provides a method of categorizing for treatment a subject suspected of having or at risk of having a neurologic disease or disorder, the method comprising performing an immunoassay to determine a concentration of Chit-1 in a biological fluid sample containing or suspected of containing the Chit-1, wherein an increased concentration of the Chit-1 in the biological fluid sample relative to a control concentration of Chit-1 obtained from a control biological fluid sample is indicative of neurologic disease or disorder in the subject and the subject is confirmed as a candidate for a neurologic treatment. The neurological disease or disorder can be for example, ALS, brain metastases, viral encephalitis, neuropathy, multiple sclerosis, upper motor neuron disease, primary lateral sclerosis, chronic inflammatory demyelinating polyneuropathy, idiopathic sensorimotor polyneuropathy, spinocerebellar ataxia, lymphoma, or lower motor neuron disease.

In one aspect, the neurologic disease is Amyotrophic Lateral Sclerosis (ALS) and the subject is confirmed as a candidate for ALS treatment.

In another aspect of the method, recovery by the immunoassay of the Chit-1 is at least 70%, at least 80%, at least 90%, or at least 95%.

In another aspect of the method, the inter-assay variability of the immunoassay is less than 11%.

In another aspect of the method, the intra-assay variability of the immunoassay is less than 6%.

The method optionally further includes determining a concentration of CHI3L1 in the biological fluid sample of the subject, wherein an increased concentration of CHI3L1 in the biological fluid sample relative to a control concentration of CHI3L1 obtained from a control biological fluid sample, is further indicative of neurological disease in the subject.

In one aspect of any of the methods, the concentration of Chit-1 and/or CHI3L1 is determined in each of a plurality of samples taken from the subject over a period of time. An increasing concentration of Chit-1 and/or CHI3L1 in the samples over the period of time is indicative of fast progressing ALS. A decreasing or constant concentration of Chit-1 and/or CHI3L1 in the samples over the period of time is indicative of slow or intermediate progressing ALS.

In one aspect, the concentration of Chit-1 and/or CHI3L1 in the sample can distinguish the subject having ALS from subjects having another neurological disease or disorder selected from the group consisting of brain metastases, viral encephalitis, neuropathy, multiple sclerosis, upper motor neuron disease, primary lateral sclerosis, chronic inflammatory demyelinating polyneuropathy, idiopathic sensorimotor polyneuropathy, spinocerebellar ataxia, lymphoma, and lower motor neuron disease.

In another aspect, the present disclosure provides a method of categorizing for treatment a subject suspected of having or at risk of having a neurological disease or disorder, the method comprising performing an immunoassay to determine a concentration of CHI3L1 in a biological fluid sample from the subject, wherein an increased concentration of the CHI3L1 in the biological fluid sample relative to a control concentration of CHI3L1 obtained from a control biological fluid sample is indicative of the presence in the subject of a neurological disease or disorder, and the subject is confirmed as a candidate for treatment of a neurological disease or disorder. In the method, the neurological disease or disorder may be ALS, brain metastases, viral encephalitis, neuropathy, multiple sclerosis, upper motor neuron disease, primary lateral sclerosis, chronic inflammatory demyelinating polyneuropathy, idiopathic sensorimotor polyneuropathy, spinocerebellar ataxia, lymphoma, or lower motor neuron disease.

In another aspect, the present disclosure provides a method of determining a treatment protocol for a subject suspected of having or at risk of having a neurological disorder such as ALS, the method comprising performing an immunoassay on a biological fluid sample obtained from the subject to determine a concentration of Chit-1 in the sample, and assigning an ALS treatment protocol to the subject if the sample contains an increased concentration of Chit-1 relative to a control concentration of Chit-1 obtained from a control biological fluid sample. The method may further comprise determining the concentration of Chit-1 in each of a plurality of samples taken from the subject over a period of time, wherein an increasing concentration of Chit-1 in the samples over the period of time is indicative of fast progressing ALS, and the method further comprises assigning a treatment protocol for fast progressing ALS to the subject; and wherein a decreasing or constant concentration of Chit-1 in the samples over the period of time is indicative of slow or intermediate progressing ALS, and the method further comprises assigning a treatment protocol for slow or intermediate progressing ALS to the subject. The immunoassay can further determine a concentration of CHI3L1 in the biological fluid sample of the subject, wherein an increased concentration of CHI3L1 in the biological fluid sample relative to a control concentration of CHI3L1 obtained from a control biological fluid sample, is further indicative of Amyotrophic Lateral Sclerosis (ALS) in the subject.

In another aspect, the present disclosure provides a method of determining a treatment protocol for a subject having a neurological disease or disorder, the method comprising performing an immunoassay on a biological fluid sample obtained from the subject to determine a concentration of CHI3L1 in the sample, and assigning a neurological treatment protocol to the subject if the sample contains an increased concentration of CHI3L1 relative to a control concentration of CHI3L1 obtained from a control biological fluid sample, wherein the neurological treatment protocol comprises administering an anti-neuroinflammatory agent to the subject.

In another aspect, the present disclosure provides a method of monitoring the therapeutic effect of an ALS treatment protocol in a subject being treated with the ALS treatment protocol, the method comprising performing an immunoassay on a first biological fluid sample obtained from the subject to determine a concentration of Chit-1 in the first sample, performing an immunoassay on a second biological fluid sample obtained from the subject at a period of time after the first biological fluid sample is obtained to determine a concentration of Chit-1 in the second sample, and comparing the concentration of Chit-1 in the first sample and the second sample, wherein a decreased or maintained concentration of Chit-1 in the second sample relative to the first sample is indicative that the treatment protocol is therapeutically effective in the subject.

In another aspect, the present disclosure provides a method of predicting ALS progression in a subject, the method comprising performing an immunoassay on a biological fluid sample obtained from the subject to determine a concentration of Chit-1 and/or CHI3L1 in the sample, wherein a baseline concentration of at least about 28 ng/ml of Chit-1, and/or a baseline concentration of at least about 390 ng/ml of CHI3L1 in the biological fluid sample, is indicative of fast progressing ALS in the subject, and a treatment protocol for fast progressing ALS is assigned to the subject.

In another aspect, the present disclosure provides a method of predicting ALS progression in a subject, the method comprising performing an immunoassay on a biological fluid sample obtained from the subject to determine a concentration of Chit-1 and/or CHI3L1 in the sample, wherein a baseline concentration of about 7.0 ng/ml or less of Chit-1, and/or a baseline concentration of about 220 ng/ml of CHI3L1 in the biological fluid sample, is indicative of slow progressing ALS in the subject, and a treatment protocol for slow progressing ALS is assigned to the subject. In one aspect, the baseline concentration of CHI3L1 is about 220 in blood or plasma, and about 200 ng/ml in CSF.

In any of the methods, the biological fluid sample can be selected from CSF, urine, saliva, blood, plasma, serum, cell extracts, and tissue extracts. In one aspect, the biological fluid sample is CSF or plasma.

In one aspect, the biological fluid sample is CSF wherein a Chit-1 concentration of at least about 28 ng/mL in the subject sample is further indicative of fast progressing ALS.

In another aspect, the biological fluid sample is CSF and a CHI3L1 concentration of at least about 390 ng/mL in the sample is further indicative of fast progressing ALS.

In any of the methods, the limit of detection for the assay is less than about 0.08 ng/m L.

Other aspects and features of the disclosure are described more thoroughly below.

### REFERENCE TO COLOR FIGURES

The application file contains at least one figure executed in color. Copies of this patent application publication with color figures will be provided by the Office upon request and payment of the necessary fee.

### BRIEF DESCRIPTION OF THE FIGURES

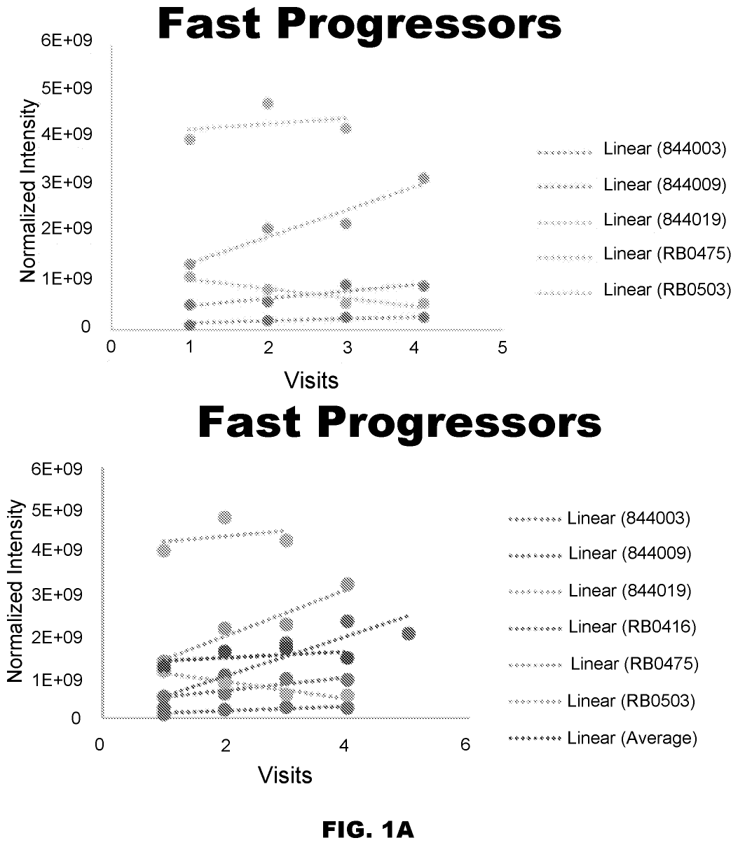

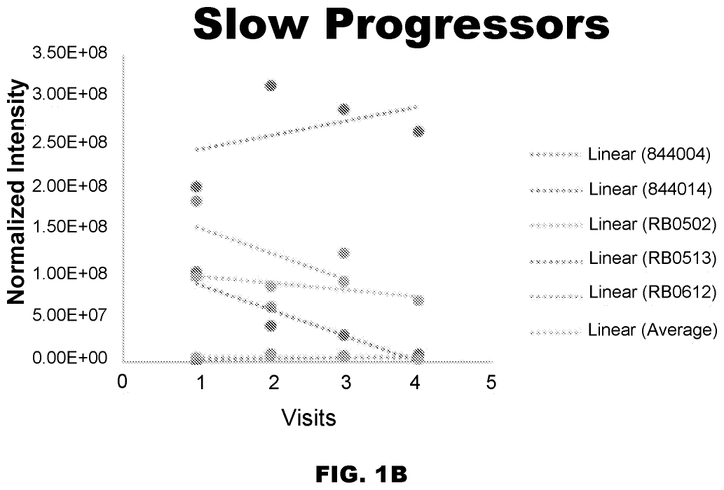

FIG. 1. Normalized Chit-1 peptide intensity in longitudinal CSF samples from fast (A) or slow (B) progressors.

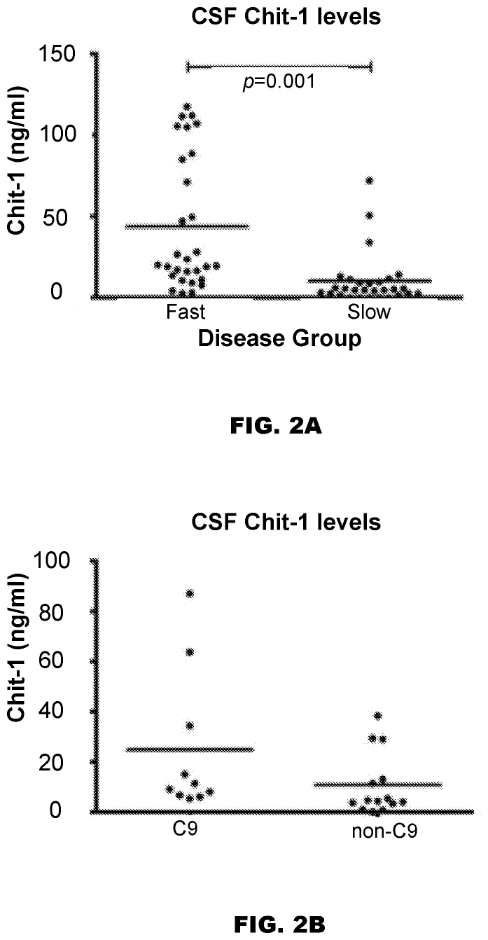

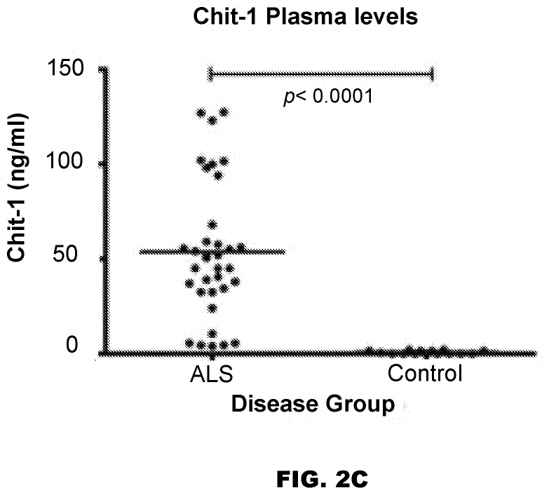

FIG. 2. Preliminary Chit-1 ELISA data from CSF and Plasma. A) Chit-1 levels in the CSF of fast versus slow progressors. Significantly higher levels occur in fast progressors (p=0.001). B) C9-ALS patients exhibited higher Chit-1 levels than a similar number of non-C9 ALS with the same disease duration. C) Plasma levels of Chit-1 were significantly higher in ALS (n=35) versus controls (n=15) (p<0.0001).

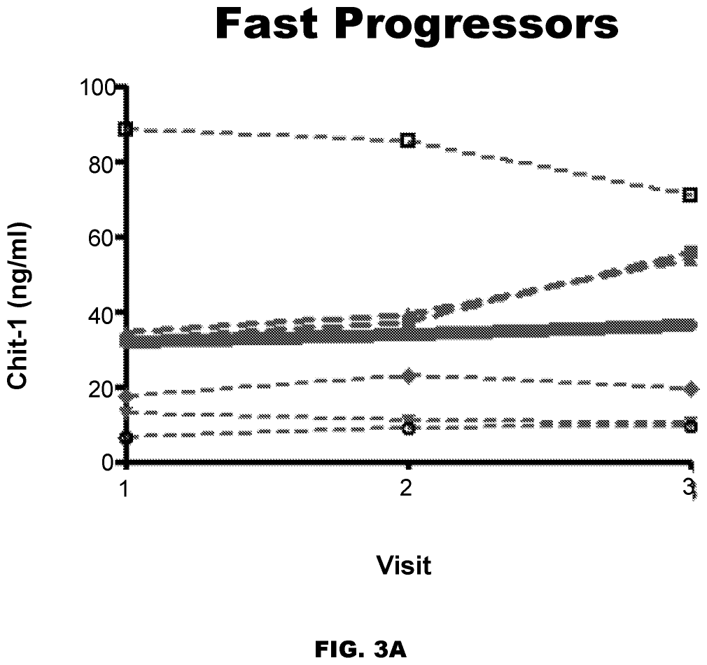

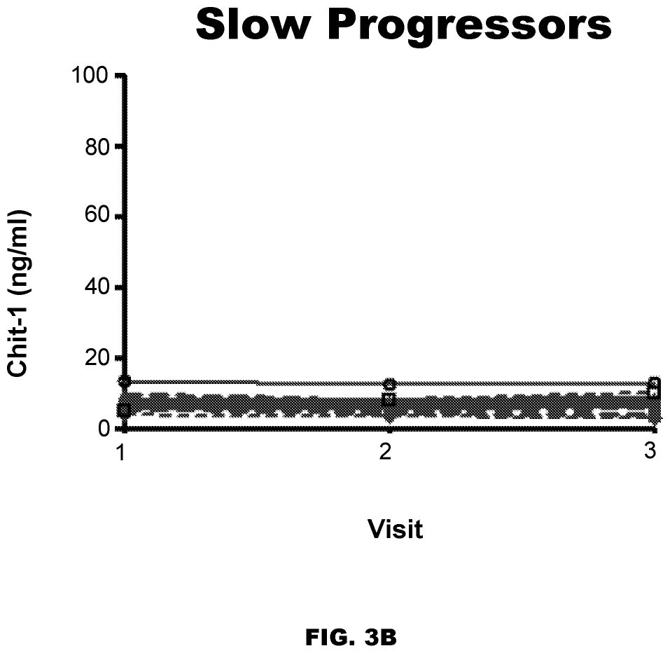

FIG. 3. Chit-1 ELISA of longitudinal CSF samples from 6 Fast progressors (Left) and 6 Slow progressors (Right). The mean of the 6 samples are shown in dark bar in each panel. Fast progressors exhibit higher levels of Chit-1 and generally increase over time. Slow progressors have lower Chit-1 levels that remain constant or slightly decrease over time.

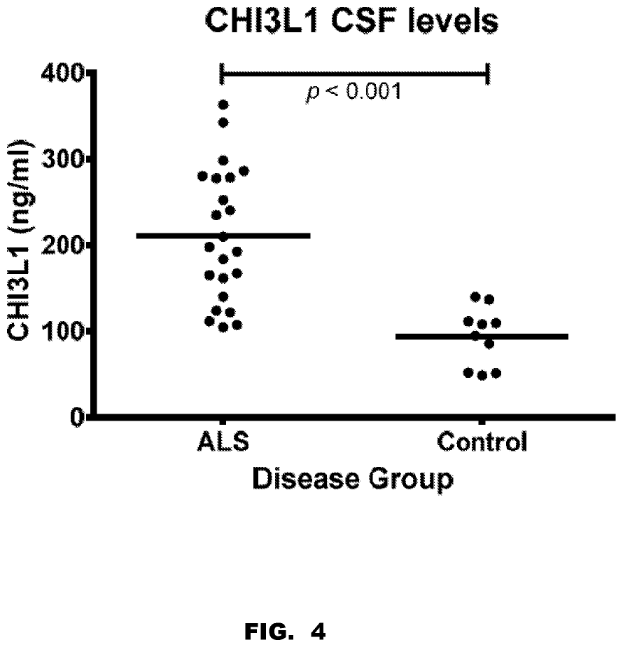

FIG. 4. CHI3L1 levels in the CSF of sporadic ALS (n=22) and healthy controls (n=10) measure by ELISA. Significantly higher CHI3L1 was detected in the CSF of ALS patients (p<0.001).

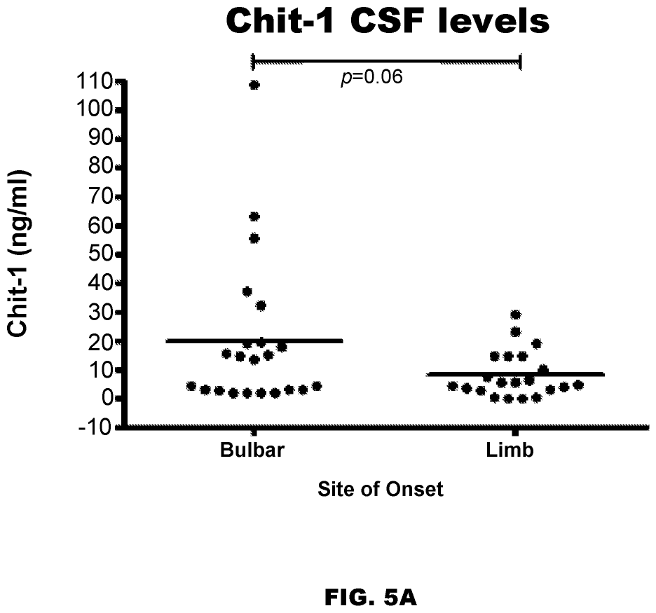

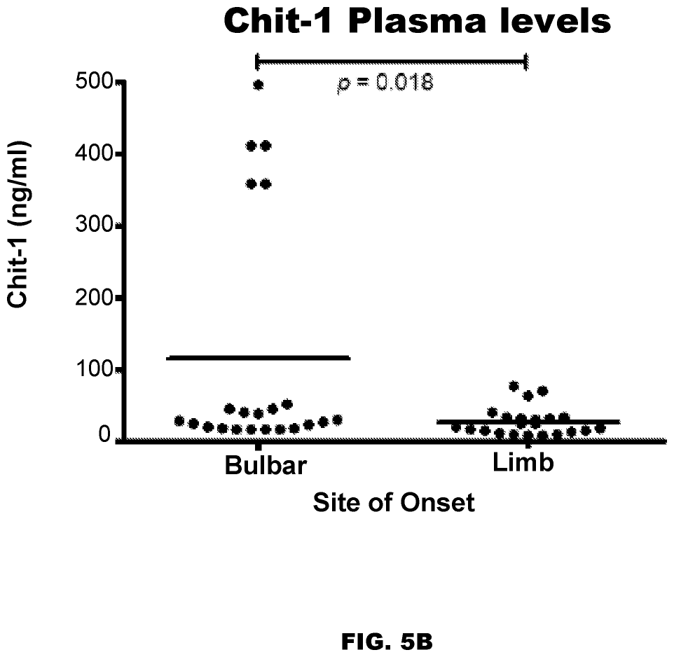

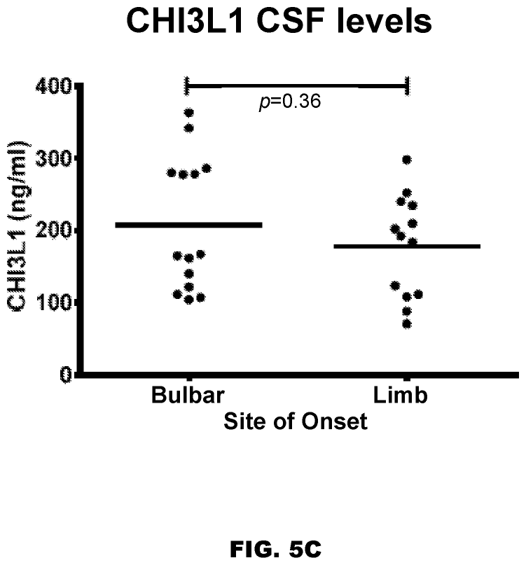

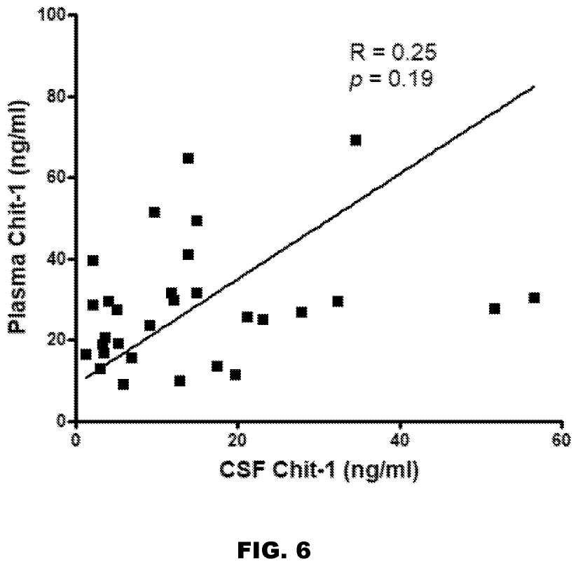

FIG. 5. Levels of Chit-1 in the CSF (A) and plasma (B) of bulbar and limb onset ALS patients (n=22). (C) CHI3L1 levels in the CSF of bulbar and limb onset ALS.

FIG. 6. Pearson correlation between CSF and plasma Chit-1 levels within the same ALS patient (n=30).

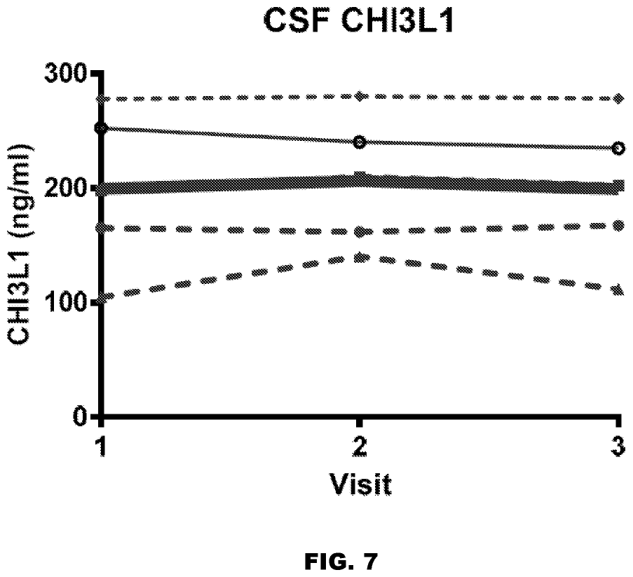

FIG. 7. CHI3L1 levels in longitudinal CSF samples from 4 slow progressors and 1 fast progressor. The Mean value is shown as a solid bar.

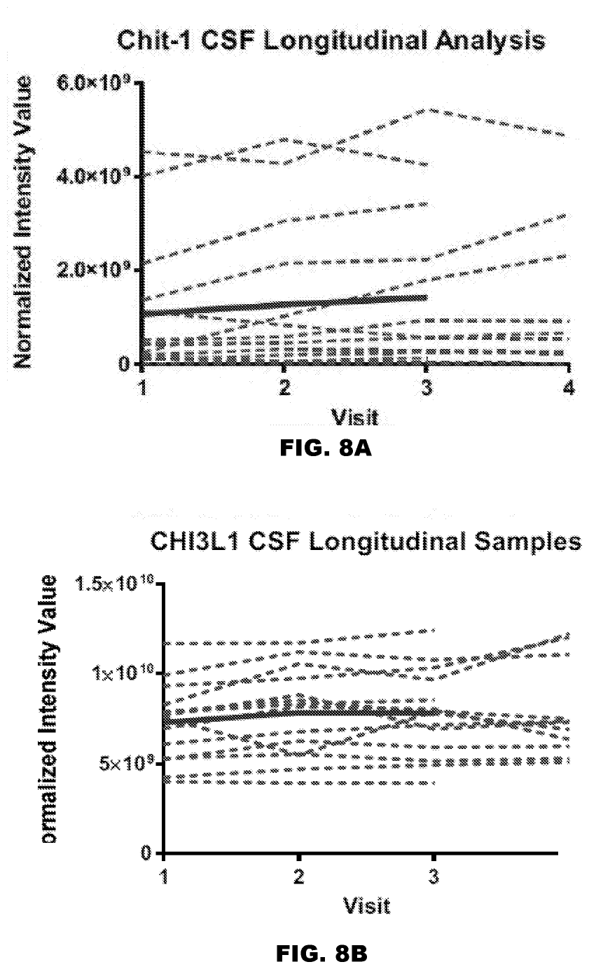

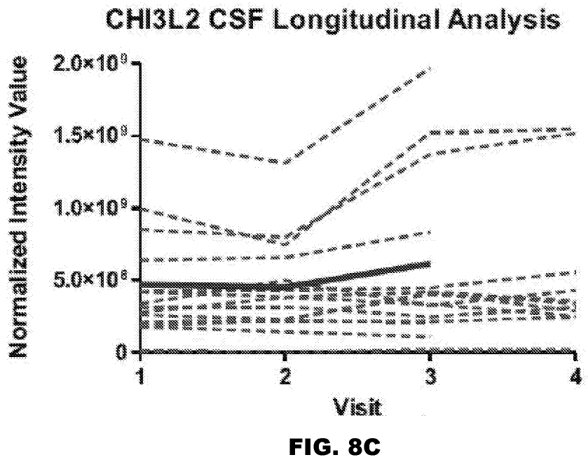

FIG. 8. Longitudinal CSF levels of chitinases by MS. Shown are the levels as represented by normalized intensity value of Chit-1 (A), CHI3L1 (B), and CHI3L2 (C) chitinases over time. Blue bar is average value over time. Visits were about every 3-4 months per patient.

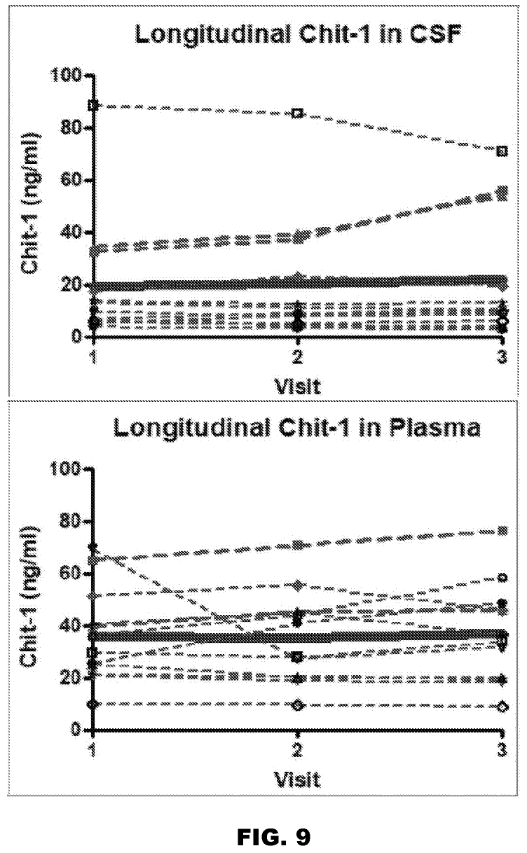

FIG. 9. Longitudinal CSF levels of Chit-1 by ELISA in CSF and Plasma samples of ALS patients. Blue bar is average value over time.

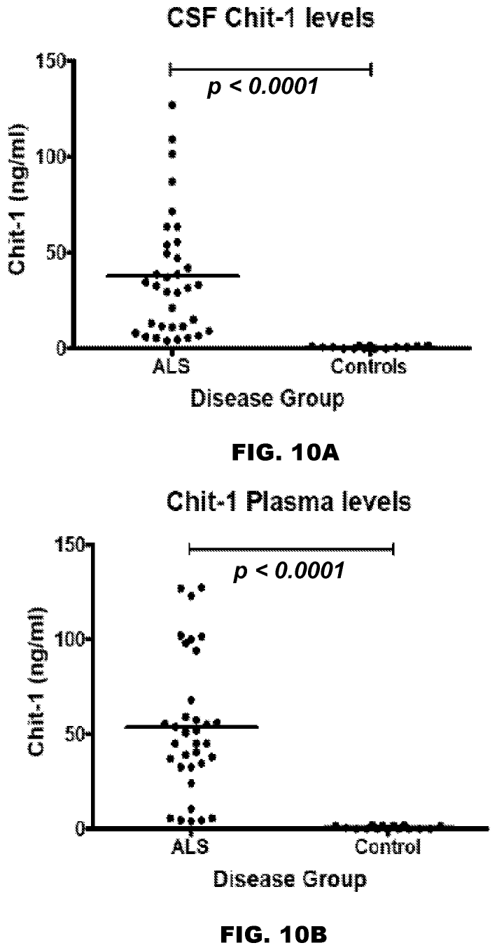

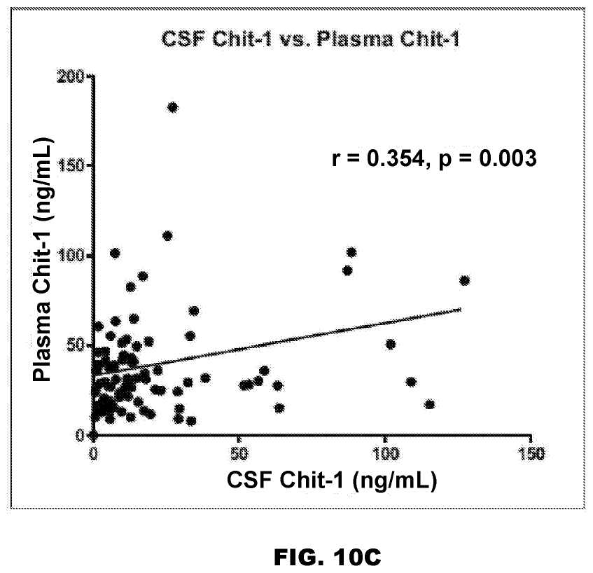

FIG. 10. Cross-Sectional Analysis of Chit-1 in CSF and Plasma. Levels of Chit-1 in CSF (A) and Plasma samples (B) of ALS patients vs. control individuals. (C) Correlation between CSF and plasma levels of Chit-1.

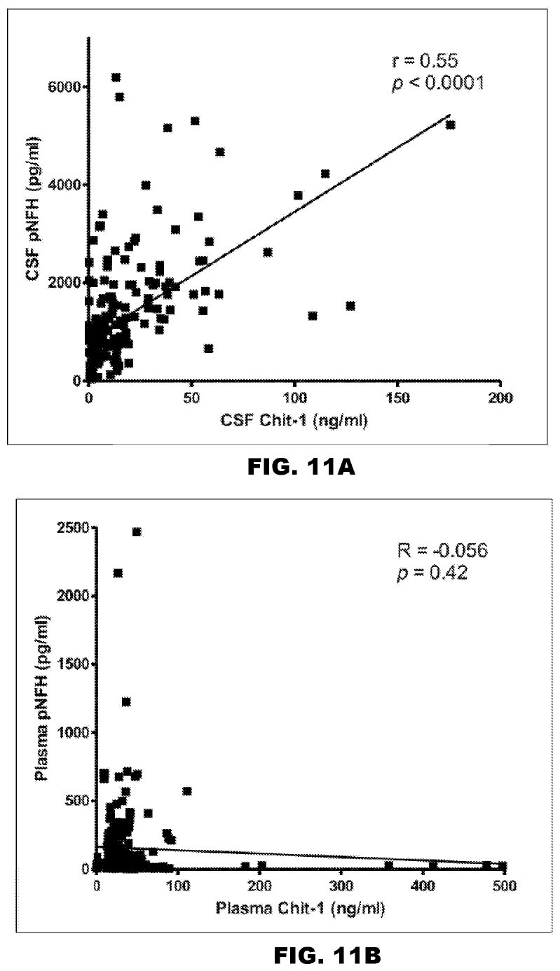

FIG. 11. Correlation of Chit-1 to pNFH in CSF and plasma. (A) Correlation in CSF. (B) Correlation in plasma.

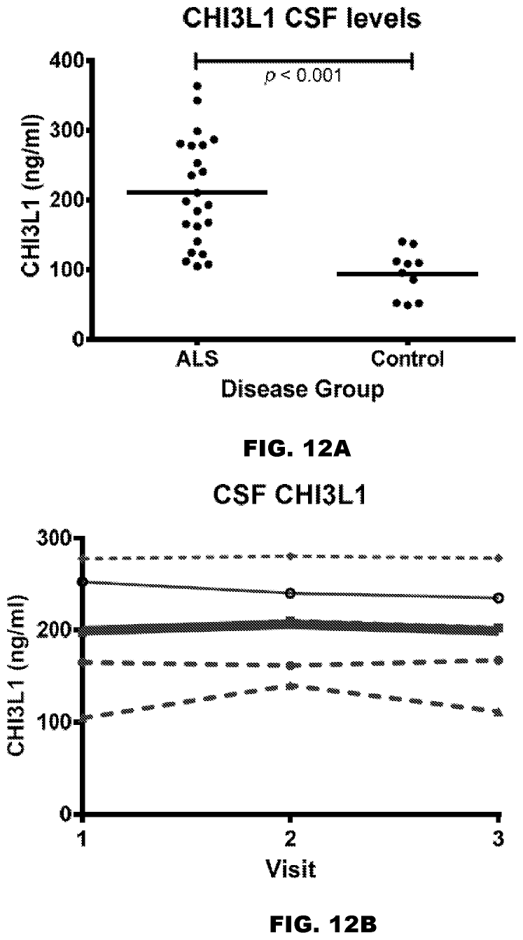

FIG. 12. (A) Levels of CHI3L1 in CSF of ALS patients and control healthy individuals. (B) Longitudinal CSF levels of CHI3L1 in CSF and Plasma samples of ALS patients. Blue bar is average value over time. Levels were measured over a period of three visits.

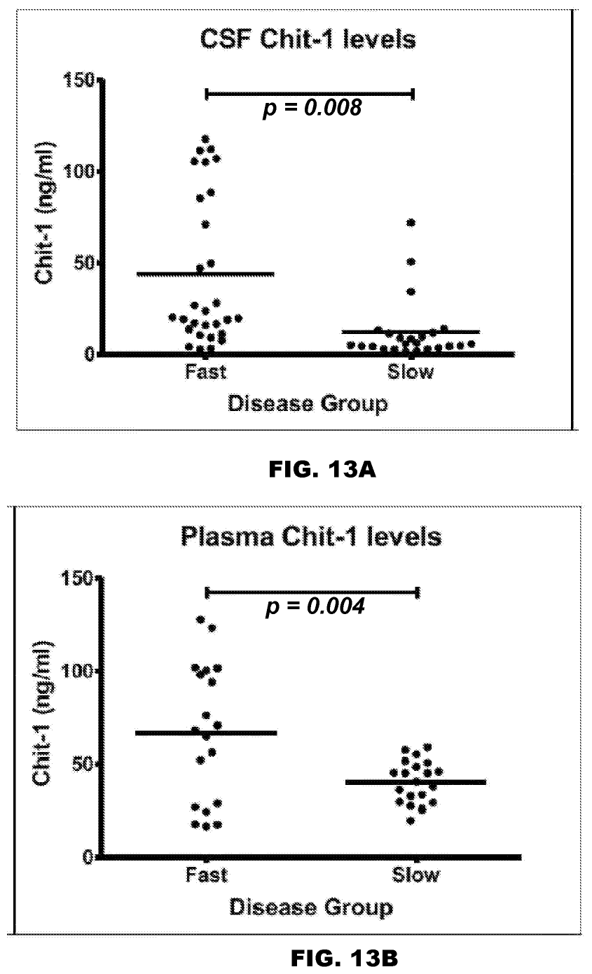

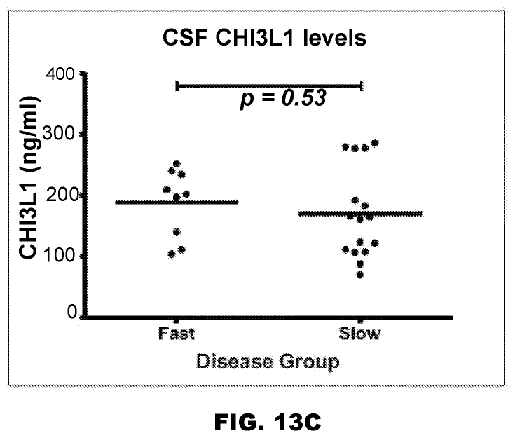

FIG. 13. Chit-1 & CHI3L1 in Fast vs. Slow Progression ALS. (A) Levels of Chit-1 in CSF. (B) Levels of Chit-1 in plasma. (C) Levels of CHI3L1 in CSF.

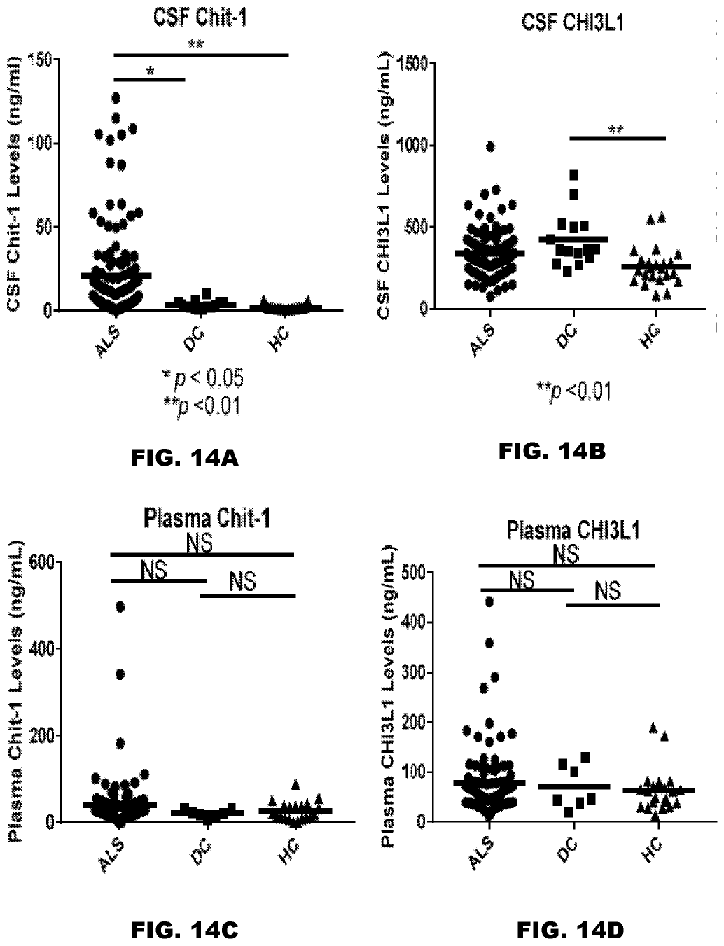

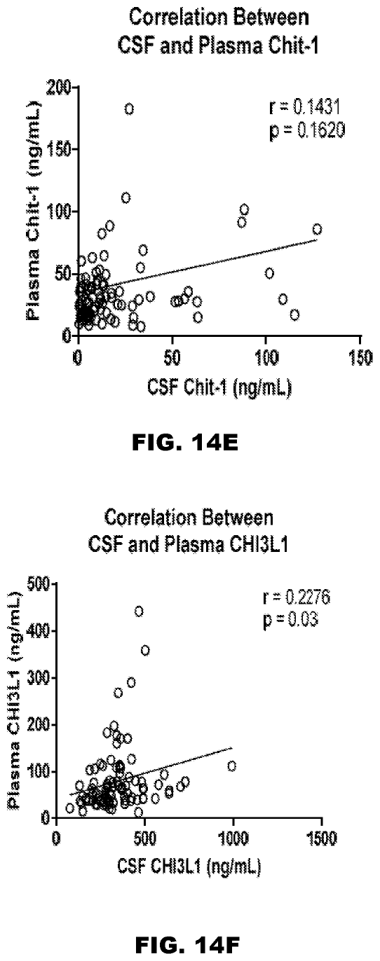

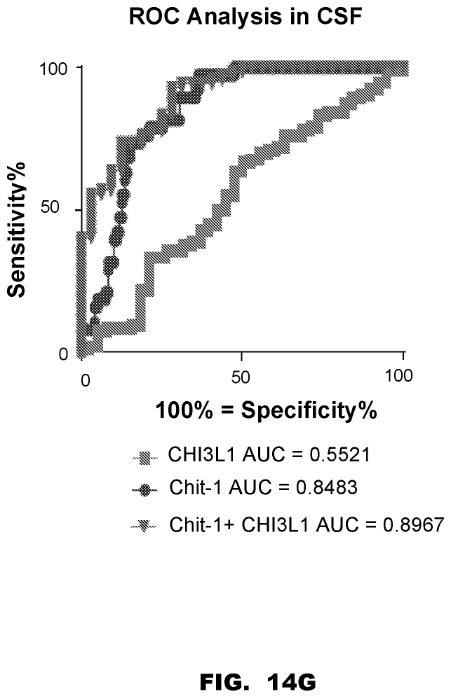

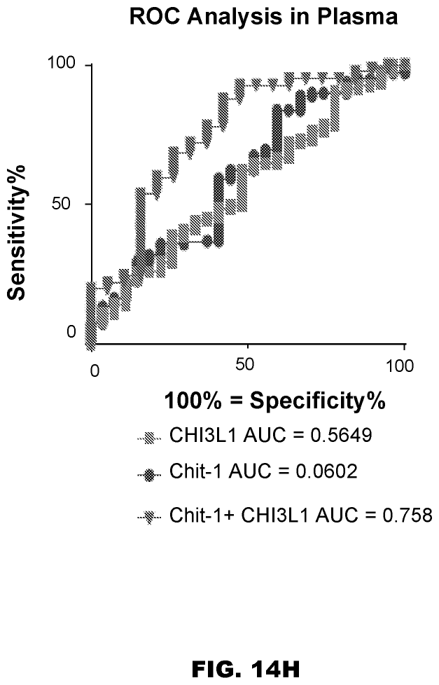

FIG. 14. Cross-sectional analysis of chitinases. (A) Levels of Chit-1 in CSF of ALS patients, patients having a range on non-ALS neurological diseases (DC), and healthy controls (HC). (B) Levels of CHI3L1 in CSF of ALS patients, patients having a range on non-ALS neurological diseases (DC), and healthy controls (HC). (C) Levels of Chit-1 in plasma of ALS patients, patients having a range on non-ALS neurological diseases (DC), and healthy controls (HC). (D) Levels of CHI3L1 in CSF of ALS patients, patients having a range on non-ALS neurological diseases (DC), and healthy controls (HC). (E) Correlation between CSF and plasma Chit-1. (F) Correlation between CSF and plasma CHI3L1. (G) ROC analysis of CSF CHI3L1 (red), CSF Chit-1 (blue), and both CSF Chit-1 and CSF CHI3L1 (purple). (H) ROC analysis of plasma CHI3L1 (red), plasma Chit-1 (blue), and both plasma Chit-1 and plasma CHI3L1 (purple).

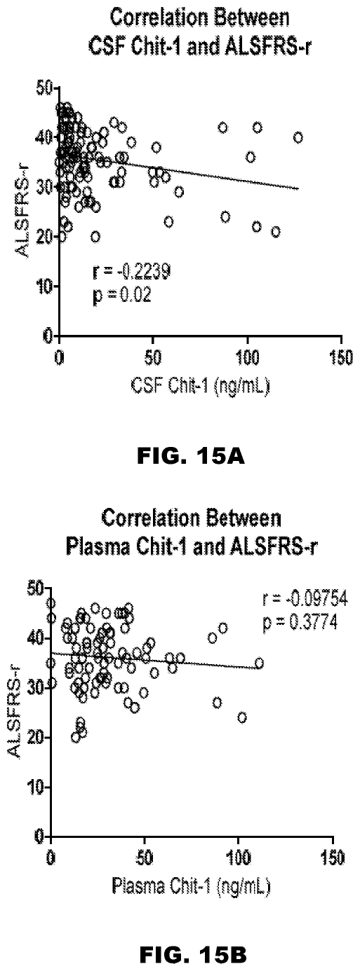

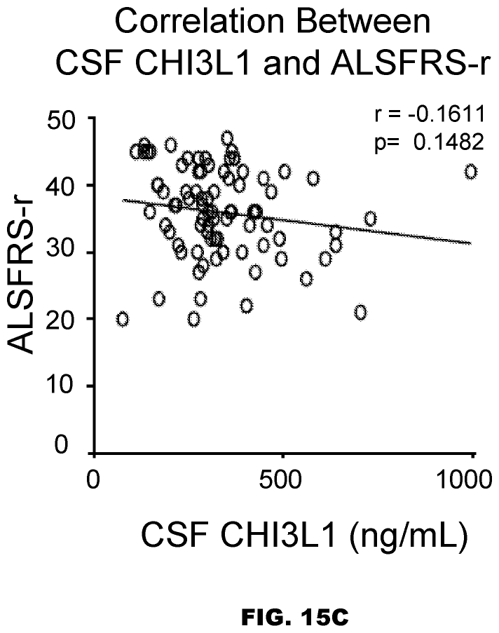

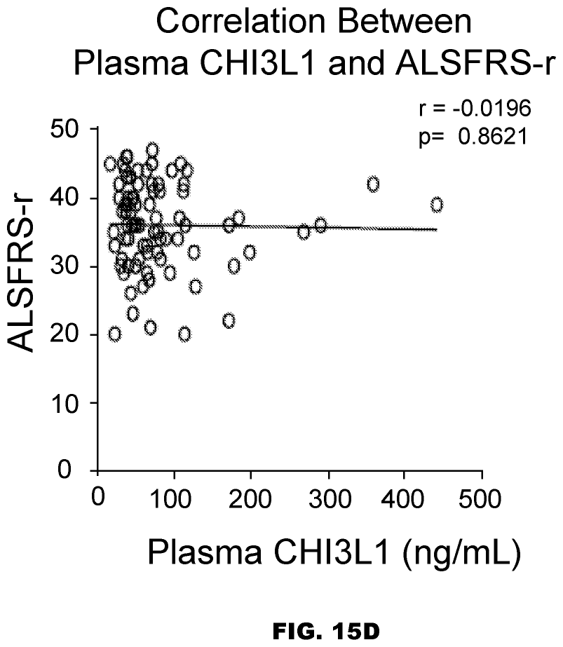

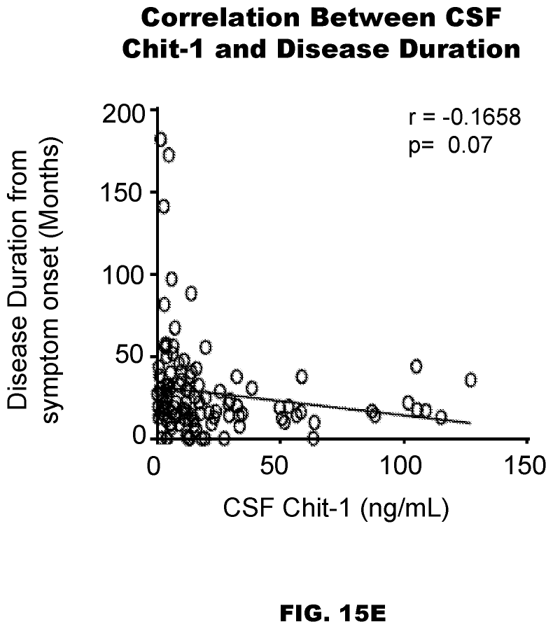

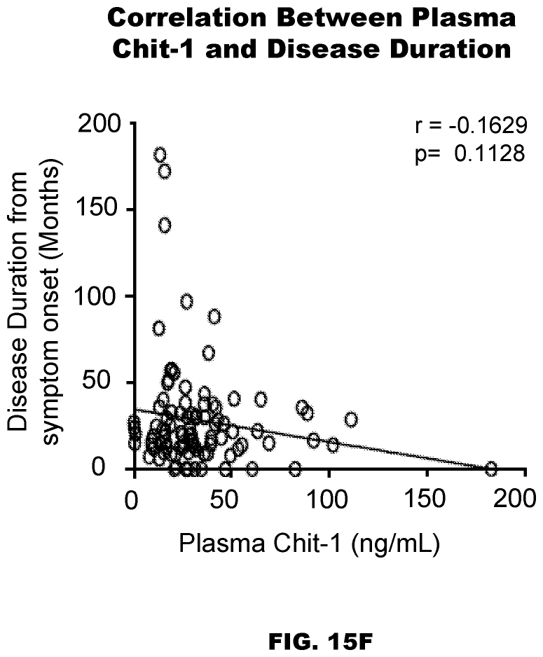

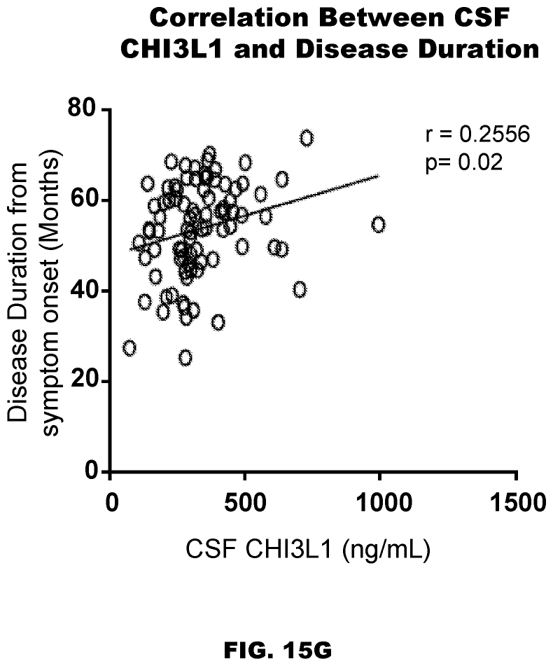

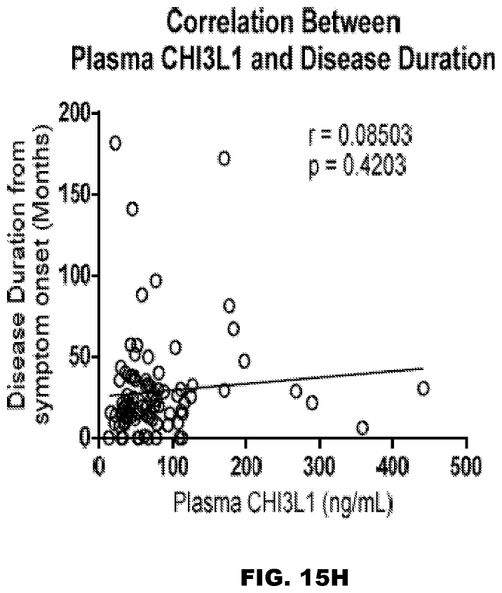

FIG. 15. Correlation with Clinical Measures of ALS. (A) Correlation between CSF Chit-1 and ALSFRS-r. (B) Correlation between plasma Chit-1 and ALSFRS-r. (C) Correlation between CSF CHI3L1 and ALSFRS-r. (D) Correlation between plasma CHI3L1 and ALSFRS-r. (E) Correlation between CSF Chit-1 and disease duration. (F) Correlation between plasma Chit-1 and disease duration. (G) Correlation between CSF CHI3L1 and disease duration. (H) Correlation between plasma CHI3L1 and disease duration.

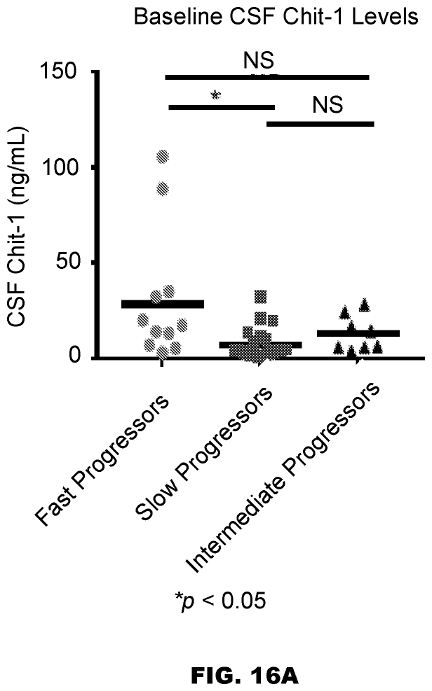

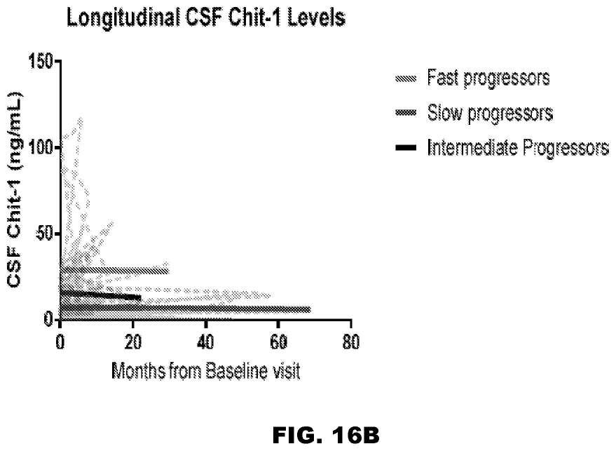

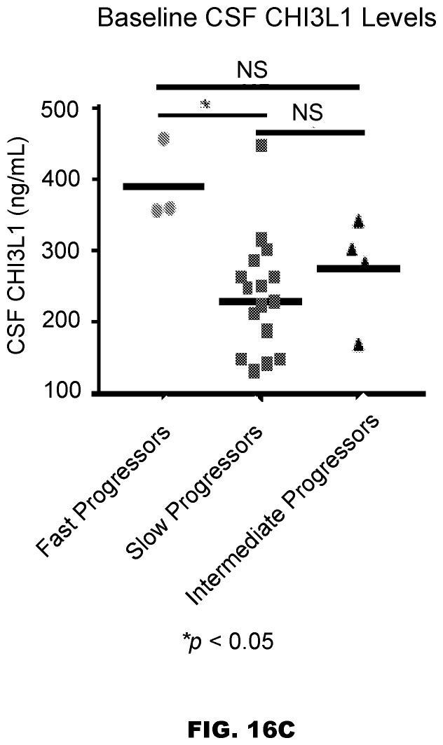

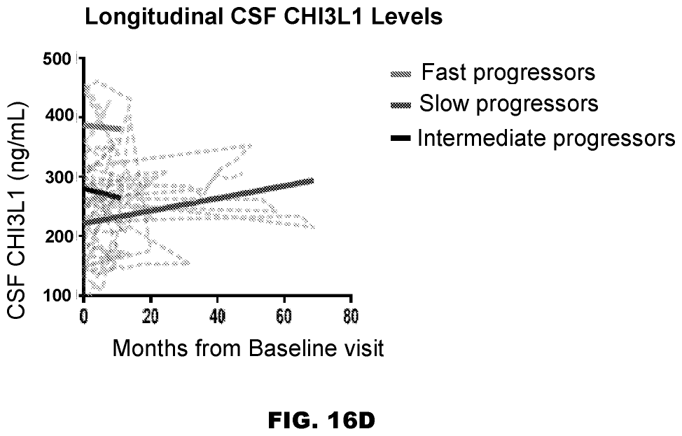

FIG. 16. Analysis of Disease Progression in CSF. (A) CSF Chit-1 levels in fast progressors (red), slow progressors (blue), and intermediate progressors (black). (B) Longitudinal CSF Chit-1 levels in fast progressors (red), slow progressors (blue), and intermediate progressors (black). (C) CSF CHI3L1 levels in fast progressors (red), slow progressors (blue), and intermediate progressors (black). (D) Longitudinal CSF CHI3L1 levels in fast progressors (red), slow progressors (blue), and intermediate progressors (black).

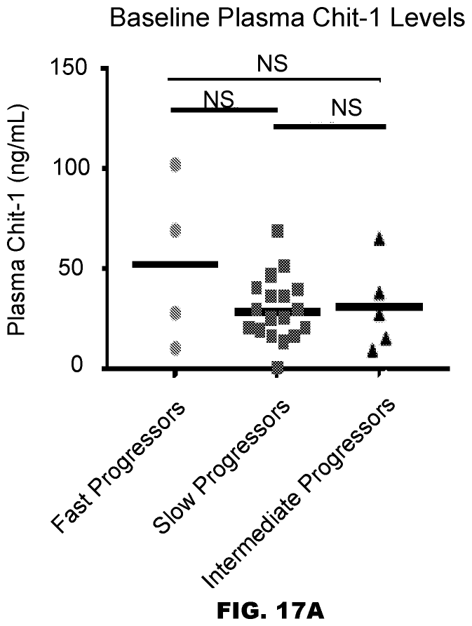

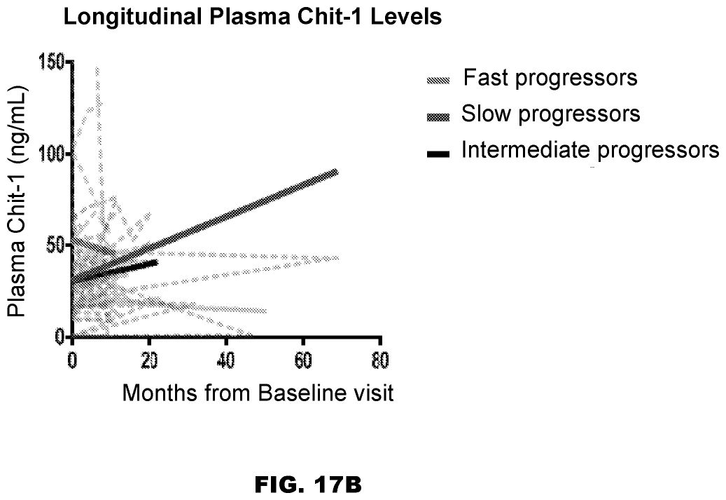

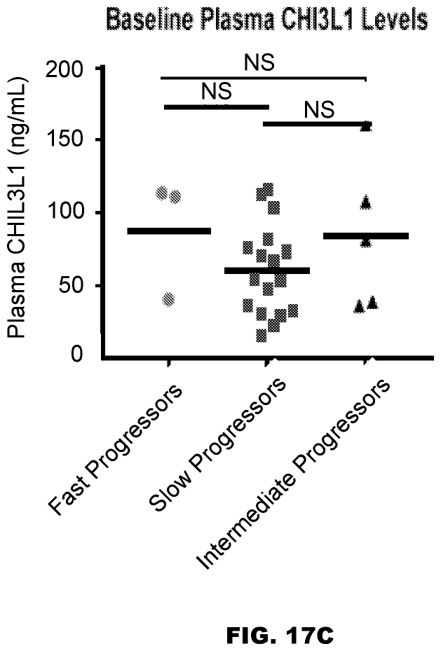

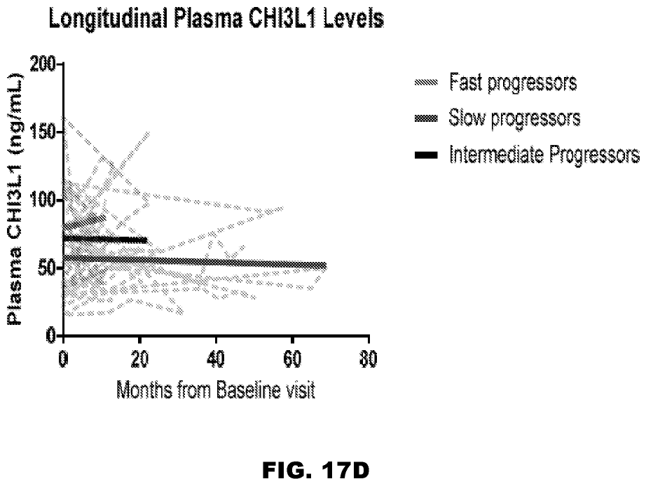

FIG. 17. Analysis of Disease Progression in plasma. (A) plasma Chit-1 levels in fast progressors (red), slow progressors (blue), and intermediate progressors (black). (B) Longitudinal plasma Chit-1 levels in fast progressors (red), slow progressors (blue), and intermediate progressors (black). (C) plasma CHI3L1 levels in fast progressors (red), slow progressors (blue), and intermediate progressors (black). (D) Longitudinal plasma CHI3L1 levels in fast progressors (red), slow progressors (blue), and intermediate progressors (black).

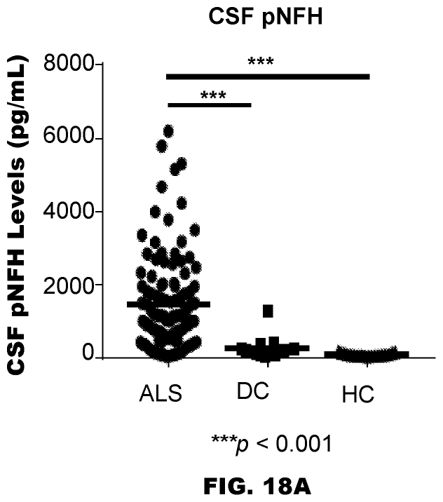

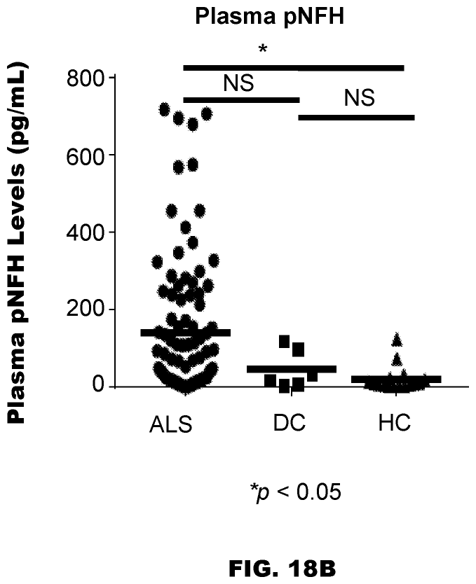

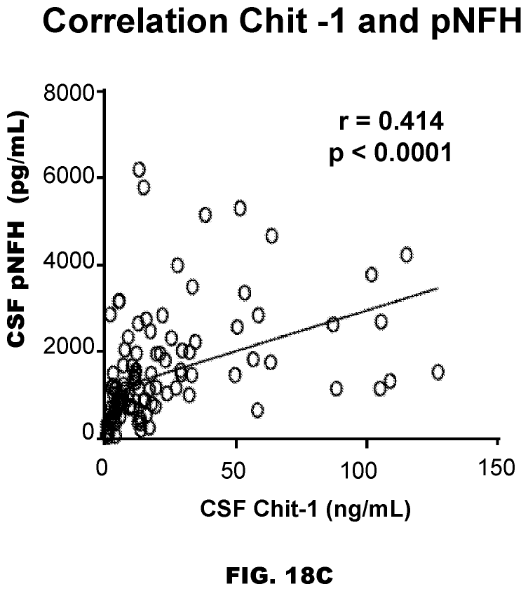

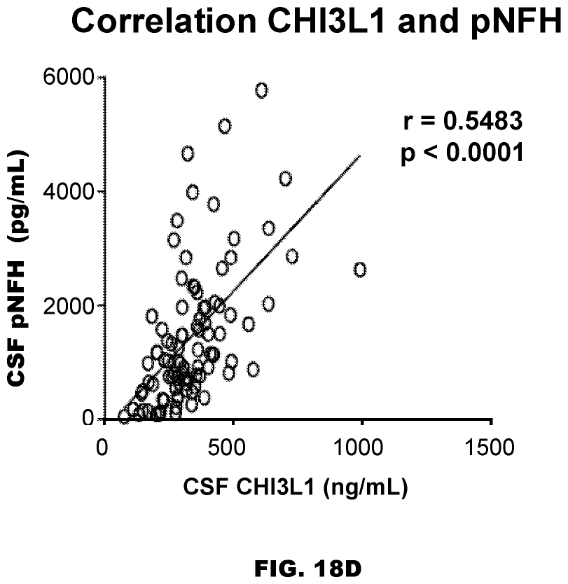

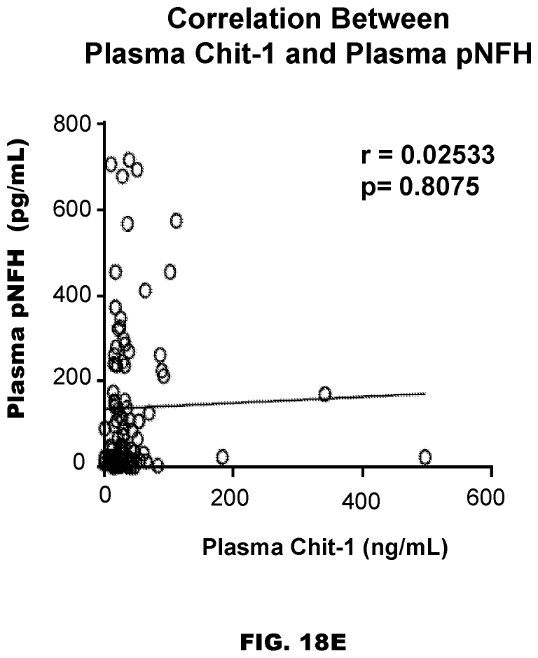

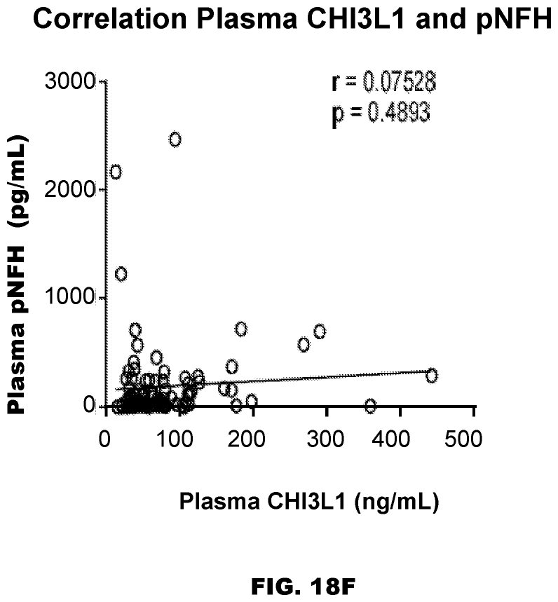

FIG. 18. Cross-Sectional Analysis of pNFH and correlation with chitinases. (A) Levels of pNFH in CSF of ALS patients, patients having a range on non-ALS neurological diseases (DC), and healthy controls (HC). (B) Levels of pNFH in plasma of ALS patients, patients having a range on non-ALS neurological diseases (DC), and healthy controls (HC). (C) Correlation between CSF Chit-1 and pNFH. (D) Correlation between CSF CHI3L1 and pNFH. (E) Correlation between plasma Chit-1 and pNFH. (D) Correlation between plasma CHI3L1 and pNFH

### DETAILED DESCRIPTION OF THE INVENTION

Chitinases are an evolutionarily conserved family of secreted proteins that bind and degrade chitin, the most abundant biopolymer in nature and an essential structural component of arthropods. Surprisingly, humans express seven chitinases including Chit-1, the first mammalian chitinase to be described. Chit-1 is expressed in activated macrophages, microglia and neutrophils and upregulated in both acute and chronic inflammatory conditions. Chitinases play pivotal roles in defense against chitin-containing human pathogens and in the homeostasis of both the innate and adaptive immune responses. Different human chitinases, including Chit-1, CHI3L1 (also known as YKL-40) and CHI3L2 are highly expressed in various inflammatory conditions and diseases, and in particular those impacting motor neurons.

The present disclosure is the first description of the use of a simple but sensitive immunoassay test that reliably and accurately determines the concentration of specific chitinases in biological fluid samples, to specifically detect macrophage/microglia and astrocyte activation and link the chitinase levels to the presence and/or progress of a neurologic disease such as ALS in a test subject. The methods disclosed herein are useful for diagnosis and prognosis, for clinical trials to stratify patient populations, and to monitor efficacy of therapies that target specific immunologic pathways and cell types.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The terms “polypeptide” and “protein” are used interchangeably to refer to a polymer of amino acid residues.

The term “neurologic disease or disorder” refers broadly to dysfunction in the CNS and/or peripheral nervous system of a mammal, caused by or related to inflammation. “Neurological disease or disorder” as used herein encompasses but is not limited to ALS, Alzheimer's disease, Parkinson's disease, brain metastases, viral encephalitis, neuropathy, multiple sclerosis, upper motor neuron disease, primary lateral sclerosis, chronic inflammatory demyelinating polyneuropathy, idiopathic sensorimotor polyneuropathy, spinocerebellar ataxia, lymphoma, and lower motor neuron disease.

The term “subject” refers to any mammal, including a human, non-human primate, dog, rat, mouse, or guinea pig which suffers, is suspected of or is at risk of suffering from a neurologic disease or disorder, whether occurring naturally or induced for experimental purposes.

The term “biological fluid” refers to any fluid produced by an organism including an animal and a cell. Non-limiting examples of a biological fluid are serum, plasma, urine, whole blood, saliva, interstitial fluid, cytosol, cell extract and tissue extract.

The term “baseline” as used with respect to a concentration of a chitinase protein in a sample, refers to the concentration of the chitinase protein present in an initial fluid sample taken from a subject suspected or at risk of suffering from a neurologic disease or disorder, prior to treatment for such neurologic disease or disorder.

### Methods

The methods described herein rely in part on the discovery of a new and improved immunoassay for determining the concentration of a chitinase protein in a fluid sample. A chitinase protein can be for example Chit-1, CHI3L1 or CHI3L2.

As noted above, different human chitinases are highly expressed in various inflammatory conditions and diseases, and in particular those impacting motor neurons. For example, neuroinflammation is a common characteristic of human subjects with ALS, manifesting as microglial activation, astrogliosis, and infiltration of monocytes and T-cells. Very early stage ALS is believed to involve the release of signals from motor neurons to microglia to release neuroprotective factors, proceeding to an imbalance leading to the disease phenotype. Thus it is believed that candidate therapies involving anti-inflammatory action may benefit subjects suffering from ALS. For example, candidate anti-inflammatory compounds that have recently been investigated for treating ALS include celecoxib, erythropoietin, glatiramer acetate, minocycline, NP001, pioglitazone and valproic acid. That these candidates have not been successful in treating ALS to date does not preclude successful candidate anti-inflammatory compounds being identified in the near future. Identifying subjects for which candidate anti-inflammatory compounds may be beneficial is thus one application of the methods disclosed herein. The methods disclosed herein provide the ability to identify early stage ALS sufferers at a relatively low cost, and painlessly and can be employed to stratify subject into slow- or moderate-progressing and fast-progressing categories. With a positive immunoassay result, a subject at a very early stage of disease may be beneficially assigned to an anti-inflammatory treatment protocol to slow progress of the disease.

The concentration of a chitinase such Chit-1, CHI3L1 or CHI3L2 in a biological fluid sample from a subject is determined using an immunoassay as described herein. A preferred immunoassay format is an enzyme linked immunosorbant assay (ELISA). ELISA is a plate-based assay technique designed for detecting and quantifying substances such as peptides, proteins, antibodies and hormones. Other names, such as enzyme immunoassay (EIA), are also used to describe the same technology. In an ELISA, an antigen is immobilized on a solid surface and then complexed with an antibody that is linked to a detection enzyme. Detection is accomplished by assessing the conjugated enzyme activity via incubation with a substrate to produce a measurable product. The most crucial element of the detection strategy is a highly specific antibody-antigen interaction.

A detection enzyme or other tag can be linked directly to a primary antibody or introduced through a secondary antibody that recognizes the primary antibody. A detection enzyme can also be linked to a protein such as streptavidin if the primary antibody is biotin labeled. Non-limiting examples of enzyme labels are horseradish peroxidase (HRP), alkaline phosphatase (AP), β-galactosidase, acetylcholinesterase and catalase. A large selection of substrates is available for performing ELISA with an HRP or AP conjugate. The choice of substrate depends upon the required assay sensitivity and the instrumentation available for signal-detection (spectrophotometer, fluorometer or luminometer), and may be determined experimentally.

ELISAs can be performed using a number of formats. Immobilization of the antigen of interest, can be accomplished by direct adsorption to the assay plate or indirectly via a capture antibody that has been attached to the plate. The antigen is then detected either directly (labeled primary antibody) or indirectly (labeled secondary antibody). A direct detection method uses a labeled primary antibody that reacts directly with the antigen. Direct detection can be performed with an antigen that is directly immobilized on the assay plate or with the capture assay format. An indirect detection method uses a labeled secondary antibody for detection. The secondary antibody has specificity for the primary antibody.

Generally, the immunoassay methods employed herein have relatively low limits of detection or of quantification as compared to commercially available ELISA kits. A well-known and highly specific ELISA is a sandwich ELISA in which the antibody is bound to a solid phase or support, which is then contacted with the sample being tested to extract the antigen from the sample by formation of a binary solid phase antibody:antigen complex. In a sandwich ELISA, the secondary antibody is generally specific for the detection primary antibody only (and not the capture antibody). Generally, this is achieved by using capture and primary antibodies from different host species (e.g., mouse IgG and rabbit IgG, respectively). After a suitable incubation period, the solid support is washed to remove the residue of the fluid sample and then contacted with a solution containing a known quantity of labeled antibody. ELISA assays measure the amount of target protein (analyte) in a biological fluid sample. Basic ELISA methods and materials such as ELISA plates, and capture and detection antibodies for performing ELISA assays of specific target analytes in fluid samples are well known and readily commercially available.

An ELISA assay may be optimized. ELISA optimization may include systematically adjusting and testing components and variables of the assay to help ensure results are robust and accurate. For instance, optimization may comprise identifying preferred components of an ELISA, such as blocking buffer, wash buffer, detection antibody, enzyme conjugate, signal detection, and other buffers and diluents, and concentrations thereof.

The term “limit of detection” (“LOD”) and “limit of quantification” (“LOQ”) adhere to their ordinary meaning in the art. LOD refers to the lowest analyte concentration likely to be reliably distinguished from background noise and at which detection is feasible, and as used herein is a signal which is three standard deviations (SD) above background noise. LOQ refers to the lowest concentration at which the analyte is reliably detected and meets predefined goals for bias and imprecision are met. Generally, as is used herein, LOQ refers to the lowest concentration above the LOD wherein the coefficient of variation (CV) of the measured concentrations less than about 20%.

The concentration of analyte (target) molecules in a fluid sample may be considered to be substantially accurately determined if the measured concentration of the target molecules in the fluid sample is within about 10% of the actual concentration of the molecules in the fluid sample. In certain aspects, the measured concentration of the target molecules in the fluid sample may be within about 5%, within about 4%, within about 3%, within about 2%, within about 1%, within about 0.5%, within about 0.4%, within about 0.3%, within about 0.2% or within about 0.1%, of the actual concentration of the biomarker molecules in the fluid sample. In some cases, the measured concentration differs from the actual concentration by no greater than about 20%, no greater than about 15%, no greater than 10%, no greater than 5%, no greater than 4%, no greater than 3%, no greater than 2%, no greater than 1%, or no greater than 0.5%. The accuracy of the assay method may be determined, in some aspects, by determining the concentration of target molecules in a fluid sample of a known concentration using the selected assay method.

In one aspect, a concentration level of a chitinase protein as determined using an ELISA can be compared to a control concentration of the chitinase protein from a healthy control subject. An increased concentration of the chitinase protein in the biological fluid sample relative to a control concentration of the chitinase protein obtained from the control biological fluid sample is indicative of a neurologic disease or disorder in the subject and the subject is confirmed as a candidate for a neurologic treatment. For example, an increased concentration of Chit-1 in the biological fluid sample relative to a control concentration of Chit-1 obtained from the control biological fluid sample is indicative of a neurologic disease or disorder in the subject and the subject is confirmed as a candidate for a neurologic treatment. The neurologic disease or disorder can be for example, any of ALS, Alzheimer's disease, Parkinson's disease, frontotemporal dementia, brain metastases, viral encephalitis, neuropathy, multiple sclerosis, upper motor neuron disease, primary lateral sclerosis, chronic inflammatory demyelinating polyneuropathy, idiopathic sensorimotor polyneuropathy, spinocerebellar ataxia, lymphoma, or lower motor neuron disease. In one aspect, the neurologic disease is ALS and the subject is confirmed as a candidate for a treatment of ALS.

In one aspect, an improved immunoassay for detecting a chitinase protein is a sandwich enzyme linked immunosorbent assay (ELISA). Improvements over commercially available ELISA's for chitinases include use of preferred capture antibodies and detection antibodies, and improved buffers, washes and washing agents. For example, a goat anti-human antibody is used as the capture antibody; mouse anti-human and goat anti-mouse IgG-HRP are used for detection; wash buffers consist of Tris, sodium chloride, ProClin 300, and BSA at a pH of 7.6; blocking buffer consists of 1×TBS+1% BSA; and an HCl solution is used to stop the peroxidase reaction. An ELISA assay incorporating the foregoing elements and directed toward measurement of Chit-1 using Goat anti-human Chit-1 (R&D systems Minneapolis, Minn. Cat #AF3559) as the capture antibody, mouse anti-human Chit-1 (R&D systems Cat #MAB3559) and goat anti-mouse IgG-HRP, is an assay that achieves a high level of recovery from CSF, i.e. at least about 95% recovery of an actual concentration in the CSF sample of 8.000 ng/mL or 0.800 ng/mL, and surprisingly about 100% recovery of protein when the actual concentration in the sample is relatively low, at 0.080 ng/mL. Thus, in one aspect a lower limit of detection for the immunoassay described herein above and as further detailed in the Examples below, with at least 95% recovery, is 0.080 ng/mL, and alternatively is 0.090 ng/mL, 0.10 ng/mL, 0.20 ng/mL, 0.30 ng/mL 0.40 ng/mL, 0.50 ng/mL, 0.60 ng/mL, 0.70 ng/mL, 0.80 ng/mL, 0.90 ng/mL, 1.0 ng/mL, 2.0 ng/mL, 3.0 ng/mL, 4.0 ng/mL, 5.0 ng/mL, 6.0 ng/mL, 7.0 ng/mL, or 8.0 ng/mL. In any of the methods, the limit of detection for the assay is about 0.08 ng/mL.

A modified Chit-1 immunoassay as disclosed herein achieves protein recovery from a biological fluid sample of least 70%, at least 80%, at least 90%, at least 95%, at least 99% or at least 100% when the at concentration of Chit-1 in the sample is at least 0.080 ng/mL. The biological fluid sample is for example CSF. The inter-assay variability of the disclosed Chit-1 immunoassay is less than 11%, and the intra-assay variability of the Chit-1 immunoassay is less than 6%.

The present disclosure further provides that a similar immunoassay approach can be used to determine a concentration of the chitinase protein CHI3L1 in a biological fluid sample of the subject, either in addition to a Chit-1 concentration in a sample, or as an alternative to determining the Chit-1 level in the sample. An increased concentration of CHI3L1 in the biological fluid sample relative to a control concentration of CHI3L1 obtained from a control biological fluid sample, is indicative of a neurological disease in the subject. When combined with determination of an elevated Chit-1 concentration in the sample, an increased concentration of CHI3L1 in the biological fluid sample relative to a control concentration of CHI3L1 obtained from a control biological fluid sample, is further indicative of a neurologic disease in the subject. The neurologic disease or disorder may be any one of ALS, Alzheimer's disease, Parkinson's disease, frontotemporal dementia, brain metastases, viral encephalitis, neuropathy, multiple sclerosis, upper motor neuron disease, primary lateral sclerosis, chronic inflammatory demyelinating polyneuropathy, idiopathic sensorimotor polyneuropathy, spinocerebellar ataxia, lymphoma, or lower motor neuron disease. In one aspect, the neurologic disease is ALS.

In another aspect, determining the concentration in a sample of Chit-1 and/or CHI3L1 as disclosed herein can be used for early diagnosis to determine a treatment protocol for a subject suspected of having or at risk of having ALS. Although current ALS treatments do not reverse the damage of ALS, they can slow the progression of symptoms and prevent complications. Thus, early detection can have a significant impact.

Treatment protocols for neurologic diseases and disorders as described herein are known and prescribed according to factors known in the art. Treatment protocols may include administration of medication(s) to alleviate primary or secondary symptoms of disease, physical therapy, occupational therapy, and nutritional support. For example, medications currently approved for the treatment of ALS are Riluzole (Rilutek), which appears to slow ALS disease progression in some subjects, and Edaravone (Radicava). Clinical studies of promising pharmaceutical candidates for the treatment of neurologic diseases and disorders such as ALS are generally ongoing worldwide. Other medications as known in the art may be prescribed to provide relief from secondary symptoms, such as muscle cramps and spasms, spasticity, fatigue, depression, sleep disruption and others.

The methods disclosed herein can also be used to stratify subjects, for example subjects suffering from, suspected of suffering from, or at risk of having a neurologic disease such as ALS. Certain neurologic diseases such as ALS manifest as slow-, moderate, or fast-progressing which may be treated differently. For example, the concentration of Chit-1 and/or CHI3L1 may be determined in each of a plurality of fluid samples taken from the subject over a period of time. The period of time may be days, weeks, months or years depending on a number of factors such as the age and condition of the subject, and the expected time course of the neurologic disease or disorder that the subject suffers from, is suspected of suffering from, or is at risk of suffering from based on medical history. Where ALS is suspected or otherwise confirmed, an increasing concentration of Chit-1 and/or CHI3L1 in the samples over the period of time is indicative of fast-progressing ALS. A decreasing or constant concentration of Chit-1 and/or CHI3L1 in the samples over the period of time is indicative of slow- or intermediate-progressing ALS.

The methods encompass monitoring the therapeutic effect of an ALS treatment protocol in a subject being treated with the ALS treatment protocol, using an immunoassay described herein. For example, the method may comprise performing an immunoassay on a first biological fluid sample obtained from the subject to determine a concentration of Chit-1 in the first sample, performing an immunoassay on a second biological fluid sample obtained from the subject at a period of time after the first biological fluid sample is obtained to determine a concentration of Chit-1 in the second sample, and comparing the concentration of Chit-1 in the first sample and the second sample, wherein a decreased or maintained concentration of Chit-1 in the second sample relative to the first sample is indicative that the treatment protocol is therapeutically effective in the subject. The method can be used for example to evaluate the efficacy of a pharmaceutical agent, such as an anti-neuroinflammatory agent being administered to the subject.

The methods also encompass predicting ALS progression in a subject, by performing an immunoassay on a biological fluid sample obtained from the subject to determine a baseline concentration of Chit-1 and/or CHI3L1 in the sample. A baseline concentration of at least about 28 ng/ml of Chit-1, and/or a baseline concentration of at least about 390 ng/ml of CHI3L1 in the biological fluid sample, is indicative of fast progressing ALS in the subject, and a treatment protocol for fast progressing ALS is assigned to the subject. Alternatively, a baseline concentration in the sample of about 7.0 ng/ml or less of Chit-1, and/or a baseline concentration in the sample of about 220 ng/ml of CHI3L1, is indicative of slow progressing ALS in the subject, and a treatment protocol for slow progressing ALS is assigned to the subject.

In any of the methods, the biological fluid sample can be CSF, urine, saliva, blood, plasma, serum, cell extract, or tissue extract. Preferably the fluid sample is CSF or plasma.

### Kits

A further aspect of the present disclosure provides immunoassay kits for conducting an immunoassay for determining the concentration of a chitinase protein in a biological fluid sample. A chitinase protein may be as described above. Preferably, the immunoassay is an enzyme-linked immunosorbent assay (ELISA), preferably a sandwich ELISA. A preferred sandwich ELISA is an improved sandwich ELISA as described above.

In some aspects, a kit is provided for performing an assay to detect a Chit-1 polypeptide in a biological sample comprising two antibodies that selectively bind to an epitope of the Chit-1 polypeptide, wherein a first antibody comprises or consists of a monoclonal antibody specific for a first Chit-1 sequence and a second antibody comprising or consisting of a monoclonal antibody specific for a second Chit-1 sequence. At least one of the antibodies may be for example be a Goat anti-human Chit-1 (R&D systems Minneapolis, Minn. Cat #AF3559) as the capture antibody. A kit may further comprise mouse anti-human Chit-1 (R&D systems Cat #MAB3559) and goat anti-mouse IgG-HRP for detection. The sample may be any biological fluid sample as disclosed herein. In one aspect, the sample is plasma. In another aspect, the sample is CSF.

A kit may further comprise wash buffer comprising Tris, sodium chloride, ProClin 300, and BSA at a pH of 7.6; blocking buffer comprising 1×TBS+1% BSA; and stop buffer comprising an HCl peroxidase solution; and combinations thereof.

As used herein, “kits” refer to a collection of elements including at least one non-standard laboratory reagent for use in the disclosed methods, in appropriate packaging, optionally containing instructions for use. A kit may further include any other components required to practice the methods, such as dry powders, concentrated solutions, or ready to use solutions. In some aspects, a kit comprises one or more containers that contain reagents for use in the methods. Containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

A kit may include instructions for testing a biological sample of a subject having or at risk of developing a neurological disease. The instructions will generally include information about the use of the kit in the disclosed methods. In other aspects, the instructions may include at least one of the following: description of possible therapies including therapeutic agents; clinical studies; and/or references. The instructions may be printed directly on the container (when present), or as a label applied to the container, or as a separate sheet, pamphlet, card, or folder supplied in or with the container.

Other aspects and features of the disclosure are described more thoroughly below.

### EXAMPLES

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Increased Levels of Chit-1, CHI3L1 and CHI3L2 in the CSF of ALS Patients

Unbiased liquid chromatography tandem mass spectrometry (LC-MS/MS) and CSF from ALS, healthy controls, disease mimics and other neurologic diseases identified Chit-1, CHI3L1, and CHI3L2 as increased in the CSF of ALS patients. Peptide counts for each chitinase protein detected in the CSF of ALS were compared to peptide counts for each chitinase protein detected in the CSF of healthy controls (HC) (Table 1). Quantification of this MS data identified increased levels of each chitinase in the CSF of ALS patients.

TABLE 1
Fold change and false discovery rate (FDR)
q-value for chitinases in ALS CSF

| Protein Name | FDR q-value | Fold change (ALS vs. HC) |
|---|---|---|
| Chit-1 | 0.08 | 2.5-fold increase |
| CHI3L1 | 0.016 | 2.1-fold increase |
| CHI3L2 | 0.01 | 2.3-fold increase |

Shown are the false discovery rate (FDR), q-value for predicting ALS versus control, and the fold increase detected in ALS CSF for each chitinase protein.

### Example 2. Levels of Chit-1, CHI3L1 and CHI3L2 in Longitudinal CSF Samples of Fast and Slow Disease Progressors

LC-MS/MS was performed on longitudinal CSF samples from a small number of fast and slow disease progressors. Fast progression was defined as a loss in the ALSFRS-r score of 1.0 unit or more per month, and slow progression was defined as a loss in the ALSFRS-r score of 0.5 units per month or less. Results indicate that CSF Chit-1 peptide levels increased over time in fast progressors and decreased over time in slow progressors (FIG. 1). Further, the normalized peptide intensity for fast progressors was 1-log order higher than slow progressors, suggesting increased levels of Chit-1 in the CSF of fast versus slow progressors.

### Example 3. Development of in-House ELISA

Mass spectrometry results described in the Examples above were validated using sandwich ELISA, focusing initial efforts on Chit-1. Commercial ELISA kits are available for Chit-1, CHI3L1 and CHI3L2. The commercial ELISA kits were used to validate the mass spectrometry data. Additionally, commercially available kits were screened to identify an optimal commercially available ELISA kit, or to identify optimal ELISA conditions and generate an in-house ELISA using commercially available antibody pairs specific to each chitinase.

An in-house sandwich ELISA was generated using commercial antibodies. The assay was used to quantify Chit-1 in CSF and plasma samples. 96 well plates of Immuno Clear standard modules well strips for ELISAs (ThermoFisher Scientific, Waltham, Mass.) were coated with goat anti-human Chit-1 (R&D systems Minneapolis, Minn. Cat #AF3559). After wells were washed and blocked (consist of 1×TBS+1% BSA), purified recombinant Chit-1 protein (R&D systems Cat #3559-GH-010) diluted in 1×TBS (20 mM Tris, 150 mM sodium chloride, 0.05% ProClin 300, 1% BSA, pH=7.6) was used to generate a standard curve from 0-10 ng/m L. CSF or plasma samples were also diluted in 1×TBS. For ALS samples, a dilution of between 1:10-1:20 was used for CSF depending on sample availability and 1:100 was used for plasma. For control samples a dilution of 1:20 was used for both CSF and plasma. Standards and samples were subsequently added to the ELISA plates and incubated at room temperature for 90 min. Mouse anti-human Chit-1 (R&D systems Cat #MAB3559) and goat anti-mouse IgG-HRP were used for detection. The peroxidase reaction was developed using 3,3′,5,5′-Tetramethylbenzidine (TMB; Sigma Aldrich, St. Louis, Mo.) and stopped using 1N hydrochloric acid (HCl).

Although commercially available assays don't provide detailed descriptions of all the components in the kit (antibodies, wash buffer compositions, blocking buffers), some of the important elements of the chit-1 assay developed in-house that differ when compared to a commercial chit-1 assay kit from MBL International Corporation include:

Goat anti-human Chit-1 (R&D systems Minneapolis, Minn. Cat #AF3559) as the capture antibody

Mouse anti-human Chit-1 (R&D systems Cat #MAB3559) and goat anti-mouse IgG-HRP were used for detection.

Wash buffer is 1×TBS; 20 mM Tris, 150 mM sodium chloride, 0.05% ProClin 300, 1% BSA, pH=7.6.

Blocking buffer is 1×TBS+1% BSA.

Stop buffer is 1N HCl when compared to 1N H2SO4 the stop buffer used in the MBL kit. The in-house ELISA was compared to the commercial chit-1 assay kit from MBL International Corporation using a spike-in recovery test using each ELISA. This step is important for optimizing an assay for quantifying the protein in the proper matrix. Both the MBL Chit-1 ELISA kit and the in-house Chit-1 ELISA generate near identical standard curve when using pure protein added to the assay diluent. The spike-in recovery tests the ability of the assay to quantify the protein in a biologic matrix (CSF, serum or plasma). Both Chit-1 ELISAs were tested using pure Chit-1 spiked into control CSF (lacking any Chit-1) at three different concentrations (80 pg/ml, 800 pg/ml, and 8000 pg/mI).

Results demonstrate that the in-house developed Chit-1 ELISA out-performs the MBL ELISA kit, at least with CSF as the matrix. The in-house Chit-1 ELISA exhibited excellent intra-assay variability (CV<6%) and inter-assay variability (CV <11%) (Table 2).

TABLE 2
Spike in recovery test of Chit-1 commercial
ELISA versus in-house ELISA

| | MBL Commercial | | In-house | |
|---|---|---|---|---|
| | Chit-1 ELISA kit | | Chit-1 ELISA | |
| | Calculated | % | Calculated | % |
| Standard in CSF | pg/ml | Recovery | pg/ml | Recovery |
| QC-1 (8000 | 5355 | 66.93 | 7653 | 95.67 |
| pg/ml) | | | | |
| QC-2 (800 | 503 | 62.83 | 760 | 95.03 |
| pg/ml) | | | | |
| QC-3 (80 | 54 | 67.17 | 84 | 104.91 |
| pg/ml) | | | | |

### Example 4. CSF Chit-1 Levels are Higher in Fast Versus Slow ALS Progressors

Data using the in-house Chit-1 ELISA demonstrated that CSF Chit-1 levels are significantly higher in fast versus slow progressors (FIG. 2A). It is also noted that C9-ALS patients (ALS patients having the C9ORF72 gene abnormality) often had higher Chit-1 CSF levels than non-C9 ALS patients (FIG. 2B). Chit-1 plasma levels were also significantly increased in ALS versus controls (FIG. 2C). The controls included healthy controls, primary lateral sclerosis, and peripheral neuropathy.

### Example 5. Chit-1 Levels in Longitudinal CSF Sample

Chit-1 levels were measured in longitudinal CSF samples from 6 fast progressors (ΔALS-FRSr of 1.0 or greater unit per month) and 6 slow progressors (ΔALS-FRSr of 0.5 or less unit per month). The results confirm the LC-MS/MS findings and demonstrate that fast progressors initially have higher levels of Chit-1 in the CSF and often increase over time (FIG. 3A), whereas slow progressors have much lower levels of Chit-1 in the CSF that remains relatively constant over time (FIG. 3B).

### Example 6. CHI3L1 is Elevated in the CSF of ALS Patients

CSF and plasma levels of CHI3L1 were measured in the CSF from 22 sporadic ALS and 10 healthy controls using a commercial ELISA kit for CHI3L1 from R&D Systems (FIG. 4). Data indicate that CHI3L1 is also elevated in the CSF of ALS patients.

### Example 7. Chit-1 and CHI3L1 Levels in Bulbar and Limb Onset Patients

Data indicated that Chit-1 and CHI3L1 are increased in the blood and CSF of ALS patients. A hypothesis that Chit-1, CHI3L1, and CHI3L2 levels are increased in the blood and CSF of specific subsets of ALS patients was tested, comparing levels in sporadic and familial forms of ALS, and correlating levels to site of disease onset, gender, overall disease duration and rate of disease progression (rate of decline in ALS-FRSr). Chit-1, CHI3L1 and CHI3L2 protein levels were measured by ELISA specific to each protein. Increased levels of Chit-1 and CHI3L2 indicated monocyte/microglia activation and increased levels of CHI3L1 indicated activation of astrocytes. These biomarkers highlighted specific cell types that function in inflammation/neuroinflammation and therefore specific pathogenic mechanisms linked to ALS.

The levels of Chit-1 and CHI3L1 in bulbar and limb onset ALS patients were measured and compared. Levels of Chit-1 and CHI3L1 in bulbar versus limb onset ALS indicates that Chit-1 levels are highest in a subset of bulbar onset patients (FIGS. 5A and 5B). While plasma levels are significantly different between bulbar and limb onset, this result was generated from a subset of the bulbar patients with highly elevated Chit-1. CSF levels did not reach statistical significance, though highest Chit-1 levels in the CSF were seen in the bulbar patients. No significant differences were detected for CHI3L1 between the disease onset sub-groups (FIG. 5C).

### Example 8. Correlation of Chit-1 in Matching CSF and Plasma Samples

The correlation for each biomarker in CSF and blood from the same individual were compared. Initial findings for Chit-1 indicate a lack of correlation between matching CSF and blood samples collected at the same time from 30 ALS patients (FIG. 6).

### Example 9. Further Cross-Sectional Analysis of Chitinases in Sporadic and Familial Forms of ALS

Simple statistics (e.g., mean, median, minimum, maximum, 25^{th} percentile, 75th percentile, standard deviation, range, interquartile range proportions) and univariate and bivariate plots (e.g., bar chart, histogram, box plots, scatterplots) is generated for each biomarker in sporadic ALS, familial ALS, bulbar onset, limb onset, C9-ALS, gender, disease duration, and monthly rate of decline in ALS-FRSr, using Prism 6.0 software.

ALS sub-populations with elevated levels of Chit-1, CHI3L1 and/or CHI3L2 in the blood and CSF are identified. Fast progressing ALS patients exhibit the highest levels of Chit-1 and CHI3L2 in the blood and CSF. Logistic regression and generalized linear model (GLM) are then used to account for clinical covariates (Site of disease status, age, gender, disease duration, ΔALS-FRSr, change in forced vital capacity over time (ΔFVC)) and the most informative biomarkers and combination of biomarkers are identified. The combination of clinical parameters and protein biomarkers provides the most informative modeling of ALS disease course and patient stratification. A lack of correlation between CSF and plasma levels of Chit-1, CHI3L1 and CHI3L2 is found, and the result is interpreted as a disconnect between inflammation and glial activation between the central nervous system and the periphery. The results provide novel data regarding correlation of peripheral or central inflammation to disease progression and survival.

Biomarkers such as Chit-1, CHI3L1 and CHI3L2 that are easily measured in biofluids and indicative of specific inflammatory cell types/pathways are quite valuable to monitor therapy to specific inflammatory targets within individual ALS patients.

### Example 10. CHI3L1 Levels in Longitudinal CSF Samples

Results in a large longitudinal cohort linked to extensive clinical information confirms the utility of Chit-1, CHI3L1, and CHI3L2 proteins as prognostic indicators of disease and support their use as pharmacodynamic biomarkers in clinical trials of drugs that target neuroinflammation, and in particular drugs that target activation of astrocytes and macrophages/microglia. Data suggested that Chit-1 is higher is fast progressors and exhibits increases of time in some fast progressors, whereas Chit-1 in slow progressors is lower and remains relatively constant over time (FIG. 3). CHI3L1 level was tested in longitudinal CSF samples from a small number of ALS patients (4 slow and 1 fast progressor) and observed that CHI3L1 levels did not change over time (FIG. 7).

The results are expanded to a total of 150 ALS patients and include matching CSF and blood from each patient.

### Example 11. Longitudinal CSF Levels of Chitinases by Mass Spectrometry

Longitudinal levels of Chit-1, CHI3L1, and CHI3L2 chitinases were measured using LC-MS/MS. Measurements were taken at the 1^{st}, 2^{nd} 3^{rd} and in most instances the 4^{th} visits. Visits were every 3-4 months for each patient. Approximately 1,100 protein were detected by this analysis, and the chitinases were among the most significantly altered between ALS and healthy controls. As shown in FIG. 8, the levels of chitinases in the CSF were relatively constant levels over time.

The longitudinal levels of Chit-1 in CSF and Plasma of ALS patients were validated using ELISA (FIG. 9). ELISA results confirmed Chit-1 levels remain relatively constant over time in both biofluids.

### Example 12. Cross-Sectional Analysis of Chit-1 in CSF and Plasma

Levels of Chit-1 in CSF and plasma were measured in ALS patients and control, healthy individuals. ALS patients included 2 primary lateral sclerosis patients and 2 motor neuropathy patients. Controls included 8 healthy individuals. Levels of Chit-1 were significantly higher in CSF (FIG. 10A) and plasma samples (FIG. 10B) of ALS patients when compared to control individuals. The levels of Chit-1 in CSF and plasma correlated (FIG. 10C).

### Example 13. Correlation of Chit-1 to pNFH in CSF and Plasma

Levels of Chit-1 in CSF and plasma were compared to levels of phosphorylated neurofilament heavy chain protein (pNFH) in both CSF and plasma samples in ALS patient. pNFH is another protein based biomarker for ALS. Interestingly, the levels of Chit-1 and pNHF correlated in CSF (FIG. 11A), but not in plasma samples (FIG. 11B).

### Example 14. CHI3L1 ELISA Validation Data

The levels of CHI3L1 in CSF were validated using ELISA. Levels of CHI3L1 were measured in ALS patients and control healthy individuals. As shown in FIG. 12(A), the levels of CHI3L1 were significantly higher in ALS patients when compared to control. Levels of CHI3L1 were also measured over time (longitudinal analysis) in ALS patients. As shown in FIG. 12(B), the levels of CHI3L1 did not change significantly over time.

### Example 15. Cross-Sectional Analysis in Fast Vs. Slow Progression ALS Patients

Levels of Chit-1 in CSF and plasma were measured in ALS patients having fast or slow progression ALS. Levels of Chit-1 were significantly higher in CSF samples from patients having fast progression ALS, when compared to levels of Chit-1 in CSF samples from patients having slow progression ALS (FIG. 13A). Similarly, levels of Chit-1 were significantly higher in plasma samples from patients having fast progression ALS, when compared to levels of Chit-1 in plasma samples from patients having slow progression ALS (FIG. 13B).

Levels of CHI3L1 in CSF were measured in ALS patients having fast or slow progression ALS. Levels of CHI3L1 in CSF from patients having fast progression ALS were equivalent to levels of CHI3L1 in CSF samples from patients having slow progression ALS (FIG. 13C). Therefore, Chit-1 differentiates Fast versus Slow progressors, whereas CHI3L1 does not.

### Example 16. Cross-Sectional Analysis of Chitinases

Levels of Chit-1 in CSF and plasma were compared to Chit-1 levels in individuals having a range on non-ALS neurological diseases (diseased control; DC) (FIG. 14). The non-ALS neurological diseases include Alzheimer's disease, Parkinson's disease, brain metastases, viral encephalitis, neuropathy, multiple sclerosis, upper motor neuron disease, primary lateral sclerosis, chronic inflammatory demyelinating polyneuropathy, idiopathic sensorimotor polyneuropathy, spinocerebellar ataxia, lymphoma, and lower motor neuron disease. The levels of CSF Chit-1 were significantly higher in ALS CSF, when compared to DC CSF (FIG. 14A). As such, CSF Chit-1 can be used for diagnostic purposes to distinguish ALS from range of diseases

Similarly, the levels of CHI3L1 in CSF and plasma were compared to CHI3L1 levels in individuals having a range on non-ALS neurological diseases (diseased control; DC). The levels of CSF CHI3L1 were significantly higher in ALS CSF, when compared to DC CSF (FIG. 14B). Therefore, CSF CHI3L1 can be used for diagnostic purposes to distinguish ALS from range of diseases.

FIGS. 14E and F. No correlation between CSF and plasma Chit-1 levels were identified, indicating that Chit-1 mediated inflammation between the periphery and central nervous systems are distinct and Chit-1 protein can monitor inflammation in these separate body regions. The correlation between CSF and plasma CHI3L1 implies that as CSF levels of CHI3L1 increases the blood levels also increase. This suggests that either fluid (CSF or plasma) can be used to determine if the subject has a neurological disease.

Using ROC curve analysis, it was determined that a combination of CSF Chit-1 and CSF CHI3L1 perform better at distinguishing ALS from both diseased controls and healthy controls when compared to using each marker alone (FIG. 14G).

### Example 17. Correlation of Levels of Chitinases with Clinical Measures of ALS

The levels of CSF and plasma Chit-1, and CSF and plasma CHI3L1 were compared to the ALS Functional Rating Scale Revised (ALSFRS-r) clinical measure of ALS (FIG. 15A-D) to determine whether correlation exists between the marker levels and clinical measures of disease progression. There is a correlation between CSF Chit-1 and ALSFRS-r, indicating that as CSF Chit-1 levels increase, the overall function of the patient decreases.

The levels of CSF and plasma Chit-1, and CSF and plasma CHI3L1 were compared to the disease duration clinical measure of ALS (FIG. 15E-H) to determine whether correlation exists between the marker levels and clinical measure of disease. There is a correlation between CSF CHI3L1 and disease duration, indicating that lower levels of CSF CHI3L1 predict longer survival of the patient.

### Example 18. Analysis of Disease Progression in CSF

CSF Chit-1 levels were measured in individuals with fast progressing ALS, slow progressing ALS, and intermediate progressing ALS. Results show that levels of CSF Chit-1 are significantly higher in fast progressing ALS, when compared to slow progressing ALS (FIG. 16A). Longitudinal CSF Chit-1 levels were also measured in individuals with fast progressing ALS, slow progressing ALS, and intermediate progressing ALS. Results show that Chit-1 levels in fast progressors (FPs) were higher than Chit-1 levels in short progressors (SPs) and intermediate progressors (IPs) (FIG. 16B).

Similarly, CSF CHI3L1 levels were measured in individuals with fast progressing ALS, slow progressing ALS, and intermediate progressing ALS. Results show that levels of CSF CHI3L1 are significantly higher in fast progressing ALS, when compared to slow progressing ALS (FIG. 16C). Longitudinal CSF CHI3L1 levels were also measured in individuals with fast progressing ALS, slow progressing ALS, and intermediate progressing ALS. Results show that CHI3L1 levels in fast progressors (FPs) were higher than CHI3L1 levels in short progressors (SPs) and intermediate progressors (IPs) (FIG. 16D).

### Example 19. Analysis of Disease Progression in Plasma

Plasma Chit-1 levels were measured in individuals with fast progressing ALS, slow progressing ALS, and intermediate progressing ALS. Results show that levels of Plasma Chit-1 are significantly higher in fast progressing ALS, when compared to slow progressing ALS (FIG. 17A). Longitudinal Plasma Chit-1 levels were also measured in individuals with fast progressing ALS, slow progressing ALS, and intermediate progressing ALS. Results show that Chit-1 levels in fast progressors (FPs) were higher than Chit-1 levels in short progressors (SPs) and intermediate progressors (IPs) (FIG. 17B).

Similarly, Plasma CHI3L1 levels were measured in individuals with fast progressing ALS, slow progressing ALS, and intermediate progressing ALS. Results show that levels of Plasma CHI3L1 are significantly higher in fast progressing ALS, when compared to slow progressing ALS (FIG. 17C). Longitudinal Plasma CHI3L1 levels were also measured in individuals with fast progressing ALS, slow progressing ALS, and intermediate progressing ALS. Results show that CHI3L1 levels in fast progressors (FPs) were higher than CHI3L1 levels in short progressors (SPs) and intermediate progressors (IPs) (FIG. 17D).

### Example 20. Cross-Sectional Analysis of pNFH and Correlation with Chitinases

Levels of pNFH in CSF of ALS patients are significantly higher than pNFH in non-ALS neurological diseases (DC), and healthy controls (HC) were measured (FIG. 18A). Levels of pNFH in plasma of ALS patients are also significantly higher than in non-ALS neurological diseases (DC), and healthy controls (HC) (FIG. 18B).

Correlation of levels of pNFH in CSF of ALS patients with CSF levels of Chit-1 in CSF of ALS patients (FIG. 18C). Correlation of levels of pNFH in CSF of ALS patients with CSF levels of CHI3L1 in CSF of ALS patients (FIG. 18D). Levels of CSF Chit-1 and CHI3L1 were significantly correlated to the CSF pNFH level in the same subject, indicating a correlation of inflammatory biomarkers to neurodegeneration biomarkers.

Similarly, levels of plasma pNFH of ALS patients, patients having a range on non-ALS neurological diseases (DC), and healthy controls (HC) were measured (FIG. 18B). The levels of pNFH in plasma distinguished between ALS patients and DC and HC individuals. Correlation of levels of pNFH in plasma of ALS patients with plasma levels of Chit-1 of ALS patients (FIG. 18E). Correlation of levels of pNFH in plasma of ALS patients with plasma levels of CHI3L1 of ALS patients (FIG. 18F). There was no direct correlation between plasma levels of Chit-1 or CHI3L1 and pNFH within the same subject, indicating that a brain derived biomarker for neurodegeneration (pNFH) when detected in the blood does not correlate to Chit-1 or CHI3L1 related inflammation in the periphery. Both Chit-1 and CHI3L1 levels in the blood denote peripheral inflammation that is independent of brain related neurodegeneration.

1. A method of categorizing for treatment a subject suspected of having or at risk of having a neurologic disease or disorder, the method comprising performing an immunoassay to determine a concentration of Chit-1 chitinase, CHI3L1 chitinase, or both in a biological fluid sample containing or suspected of containing Chit-1, CHI3L1, or both, wherein an increased concentration of Chit-1 in the biological fluid sample relative to a control concentration of Chit-1, CHI3L1, or both obtained from a control biological fluid sample is indicative of neurologic disease or disorder in the subject and the subject is confirmed as a candidate for a neurologic treatment.

2. The method of claim 1, wherein the neurological disease or disorder is selected from the group consisting of ALS, Alzheimer's disease, Parkinson's disease brain metastases, viral encephalitis, neuropathy, multiple sclerosis, upper motor neuron disease, primary lateral sclerosis, chronic inflammatory demyelinating polyneuropathy, idiopathic sensorimotor polyneuropathy, spinocerebellar ataxia, lymphoma, and lower motor neuron disease.

3. (canceled)

4. The method of claim 1, wherein recovery by the immunoassay to determine the concentration of Chit-1 is at least 70%, at least 80%, at least 90%, or at least 95%.

5. The method of claim 1, wherein inter-assay variability of the immunoassay to determine the concentration of Chit-1 is less than 11%.

6. The method of claim 1, wherein intra-assay variability of the immunoassay to determine the concentration of Chit-1 is less than 6%.

7. (canceled)

8. The method of claim 1, wherein the concentration of any of Chit-1 and CHI3L1 is determined in each of a plurality of samples taken from the subject over a period of time.

9. The method of claim 8, wherein the neurologic disease is Amyotrophic Lateral Sclerosis (ALS).

10. The method of claim 9, wherein an increasing concentration of Chit-1 or CHI3L1 in the samples over the period of time is indicative of fast progressing ALS.

11. The method of claim 9, wherein a decreasing or constant concentration of Chit-1 or CHI3L1 in the samples over the period of time is indicative of slow or intermediate progressing ALS.

12. The method of claim 2, wherein the method further distinguishes the subject having ALS from subjects having a neurological disease or disorder selected from the group consisting of Alzheimer's disease, Parkinson's disease, frontotemporal dementia, brain metastases, viral encephalitis, neuropathy, multiple sclerosis, upper motor neuron disease, primary lateral sclerosis, chronic inflammatory demyelinating polyneuropathy, idiopathic sensorimotor polyneuropathy, spinocerebellar ataxia, lymphoma, and lower motor neuron disease based on absolute levels of chitinase in the biological fluid.

13-14. (canceled)

15. A method of determining a treatment protocol for a subject suspected of having or at risk of having ALS, the method comprising performing an immunoassay on a biological fluid sample obtained from the subject to determine a concentration of Chit-1 chitinase, CHI3L1 chitinase, or both in the sample, and assigning an ALS treatment protocol to the subject if the sample contains an increased concentration of Chit-1, CHIT3L1, or both relative to a control concentration of Chit-1, CHIT3L1, or both obtained from a control biological fluid sample.

16. The method of claim 15, further comprising determining the concentration of Chit-1 in each of a plurality of samples taken from the subject over a period of time.

17. The method of claim 16, wherein an increasing concentration of Chit-1 in the samples over the period of time is indicative of fast progressing ALS, and the method further comprises assigning a treatment protocol for fast progressing ALS to the subject.

18. The method of claim 16, wherein a decreasing or constant concentration of Chit-1 in the samples over the period of time is indicative of slow or intermediate progressing ALS, and the method further comprises assigning a treatment protocol for slow or intermediate progressing ALS to the subject.

19. The method of claim 15, wherein the immunoassay further determines a concentration of CHI3L1 in the biological fluid sample of the subject, wherein an increased concentration of CHI3L1 in the biological fluid sample relative to a control concentration of CHI3L1 obtained from a control biological fluid sample, is further indicative of Amyotrophic Lateral Sclerosis (ALS) in the subject.

20. The method of claim 19, wherein the biological fluid sample is CSF and wherein a CHI3L1 concentration of at least about 390 ng/mL in the subject sample is further indicative of fast progressing ALS.

21. The method of claim 1, wherein if the sample contains an increased concentration of CHI3L1 relative to a control concentration of CHI3L1 obtained from a control biological fluid sample, the subject is assigned a neurological treatment protocol, the neurological treatment protocol comprising administering an anti-neuroinflammatory agent to the subject.

22. A method of monitoring the therapeutic effect of an ALS treatment protocol in a subject being treated with the ALS treatment protocol, the method comprising performing an immunoassay on a first biological fluid sample obtained from the subject to determine a concentration of Chit-1 in the first sample, performing an immunoassay on a second biological fluid sample obtained from the subject at a period of time after the first biological fluid sample is obtained to determine a concentration of Chit-1 in the second sample, and comparing the concentration of Chit-1 in the first sample and the second sample, wherein a decreased or maintained concentration of Chit-1 in the second sample relative to the first sample is indicative that the treatment protocol is therapeutically effective in the subject.

23. A method of predicting ALS progression in a subject, the method comprising performing an immunoassay on a biological fluid sample obtained from the subject to determine a concentration of Chit-1 and/or CHI3L1 in the sample, wherein a baseline concentration of at least about 28 ng/ml of Chit-1 or a baseline concentration of at least about 390 ng/ml of CHI3L1 in the biological fluid sample, is indicative of fast progressing ALS in the subject, and a treatment protocol for fast progressing ALS is assigned to the subject, and wherein a baseline concentration of about 7.0 ng/ml or less of Chit-1 or a baseline concentration of about 220 ng/ml of CHI3L1 in the biological fluid sample, is indicative of slow progressing ALS in the subject, and a treatment protocol for slow progressing ALS is assigned to the subject.

24-25. (canceled)

26. The method of claim 1, wherein the biological fluid sample is CSF or plasma.

27. The method of claim 26, wherein the biological fluid sample is CSF and wherein a Chit-1 concentration of at least about 28 ng/mL in the subject sample is further indicative of fast progressing ALS.

28. (canceled)
